(19) United States
(12) Patent Application Publication
(10) Pub. No.: US 2025/0292881 A1
(43) Pub. Date: Sep. 18, 2025

(54) SYSTEMS AND METHODS FOR EXTRACTING DATA STRUCTURES IN NETWORK ENVIRONMENTS TO GENERATE INSTRUCTIONS

(71) Applicant: MOLAR SOLUTIONS, INC. d/b/a COUNTER HEALTH, New York, NY (US)

(72) Inventors: Rabi Alam, New York, NY (US); Rafael E. Dodo, New York, NY (US); Tyler Hicks-Wright, New York, NY (US); Giovanni Vocale, New York, NY (US); Jonathon DiTroia, New York, NY (US)

(73) Assignee: MOLAR SOLUTIONS, INC. d/b/a COUNTER HEALTH, New York, NY (US)

(21) Appl. No.: 19/077,823

(22) Filed: Mar. 12, 2025

Related U.S. Application Data
(60) Provisional application No. 63/564,372, filed on Mar. 12, 2024.

(51) Int. Cl. G16H 10/60; G06F 16/25; G16H 80/00

(52) U.S. Cl. CPC G16H 10/60; G06F 16/258; G16H 80/00

(57) ABSTRACT

Presented herein are systems and methods for aggregating claims data. A database query service may aggregate claims data from patients, care providers, pharmacy services, and a multitude of other entities to store and maintain on a centralized database. The claims data may be stored and maintained as one or more data structures in accordance with a standard template across the database to facilitate access by the entities using the database. The claims data may also identify information for entities available for provision to address health conditions of patients. The service may also establish a communication session to facilitate exchange of messages through an interface between a patient and the entities. The service may monitor for usage of an electronic card at the pharmacy service.

### CROSS REFERENCE TO RELATED APPLICATIONS

The present application claims the benefit of and priority to U.S. Provisional Patent Application No. 63/564,372, filed Mar. 12, 2024, which is incorporated by reference in its entirety.

### TECHNICAL FIELD

This application generally relates to querying databases. In particular, the present application relates to querying databases of claims data to extract information.

### BACKGROUND

Electronic records containing claims-related data may be stored and maintained across a multitude of databases. The claims data may include information on patients receiving healthcare services or prescriptions. It may be difficult to store and maintain electronic records on databases. First, the sheer volume of data may be massive, with electronic records containing a wide variety of facets of information related to the patient in receiving care from different healthcare providers and prescriptions from various pharmacies. Second, the claim data themselves may be tracked by different entities (e.g., care providers, insurers, and pharmacies) on separate databases with little to no direct integration (e.g., no communication or standard format) with one another.

Furthermore, the querying and retrieval of claims data from a massive electronic data may present its own set of challenges. One approach to querying and searching claims data may include collaborative filtering using information about the patient. Under collaborative filtering, claims data may be selected using the information about other patients that are similar (e.g., same condition or prescription) to the patient for which the query is made. This technique, however, may be inadequate in querying and selecting claims data in this context for a number of reasons. For one, data gathered about the patients may be sparse or non-existent, leading to difficulty in finding similar patients with information to be used to select claims data. For another, the selection under collaborative filtering may lack any considerations of heuristics particular to electronic records regarding healthcare services or prescriptions.

These and other factors may lead to barriers to accessing such data across different entities as well as incomplete and inconsistent entries in the claim data, making it difficult to access and query the claims data. As a consequence, there may result in time and effort spent by various entities maintaining the claims data on their databases. Even if techniques such as collaborative filtering are used to find claims data, the selected claims data may be irrelevant and not useful for the patients. This may lead to significant degradation in the delivery of care and prescriptions to patients, especially in consideration that certain medications are not to be taken with one another due to harm from potential side effects to patient. In addition, this convoluted and patchwork setup may cause wasted computer resources and network bandwidth from attempting to access and query for claims data stored across different databases.

### SUMMARY

To address these and other technical challenges, a database query service may aggregate claims data from patients, care providers, pharmacy services, and a multitude of other entities to store and maintain on a centralized database. The claims data may be stored and maintained as one or more data structures in accordance with a standard template across the database to facilitate access by the entities using the database. The claims data may also identify information for prescriptions from pharmacy services (and other entities) available for provision to address health conditions of patients. For instance, the claims data may represent available inventory of drugs for patients at a particular pharmacy service or location, as an alternative substitute for drugs prescribed for a particular patient. The claims data may identify an available prescription aimed at addressing a particular condition; a value at which to transact to receive the prescription and a pharmacy service from which to obtain the prescription.

In conjunction with the aggregation of more claim data onto the database, the service may receive a query for claims data for a patient to find alternative claims data. The query may identify a claim data initially assigned to the patient and may identify: a prescription originally designated for the patient aimed at addressing the patient's condition; a value at which to transact for the prescription; and a pharmacy service from which to obtain the prescription. Upon receipt, the service may search the claims database for candidate claims data with correspondence with the claim data of the query. The correspondence may be defined in accordance with a rule for therapeutic equivalence or generic relation. For example, the candidate claims data found may identify prescriptions that are directed to addressing the same health condition or may be a generic version of the drug identified in the query. With the identification, the service may select one of the candidate claims data as an alternate claim data to provide to the patient. The alternate claim data may have a value less than the originally assigned value as identified in the claim data of the query.

With the selection of the alternate claim data, the service may use data mining to generate a program to switch the patient from the originally assigned claim data to the selected alternate claim data. The service may collect or aggregate information about the patient, the original claim, and the alternate claim data, among others. For example, the information may include or identify historical data about: the patient, such as receiving prescriptions, refilling prescriptions, visits to a location associated with a pharmacy service or a care service provider, and adherence to a prescription; the claims, such as the condition to be addressed, the prescription, switching of claims, and the identification of the pharmacy service provider; the pharmacy service provider identifying rate of fulfillment of prescription orders, among others. The aggregation of the information may be independent of the selection of the claims data.

The service may execute a data model to information about the patient, the original claim data, and the alternate claim data, among others. The data model may be a function, a statistical model, or a machine learning (ML) model, among others. The data model may be generated and updated using heuristics regarding the information about the patient, the original claim data, and the alternate claim data. From executing the data model, the service may generate the program identifying a value likely to induce the patient to switch over from the original claim data to the alternate claim data. The program may be an executable (e.g., an application, extension, or smart contract) to be used to switch claims data for the patient and a value for the re-assignment of claims data. The program may also define a distribution schedule for assigning or transferring the value from a service provider to accounts associated with the patient across a specified period of time.

The service may return a notification identifying the selected, alternate claim data as well as the program to the patient for presentation through an application on a patient's device. The patient may be presented with an option to accept or reject the program to switch from the original claim data to alternative claim data. If the patient indicates rejection, the service may continue to monitor for alternative claims data, as more and more claims data are aggregated onto the database. Otherwise, if the patient indicates acceptance, the service may proceed with updating the patient's profile to switch from the original claim to the alternative claim data. In addition, the patient's device may store and install the program as an executable, an applet, a plug-in, or a smart contract, among others. Using the alternative claim data, the service may generate information for an electronic (or virtual) card to be used for the transaction to receive the substitute prescription. Once generated, the service may provide the electronic card information to the patient for use to receive the prescription through the pharmacy identified in the alternative claim data. The service may also listen for data associated with the patient and the claims to determine whether to transfer the value in accordance with the distribution schedule.

In connection with the query, the service may establish a communication session to facilitate exchange of messages through an interface between a patient or other stakeholders operating on behalf of the patient (e.g., family members, the service described herein, other care or service providers) and other involved entities, such as a care provider or a pharmacy service. The service may deliver the alternative claim data using a message object for the messaging interface on the patient's end-user device. The service may also provide indications of modifications to prescriptions for the patient through the messaging interface. The message object may specify field-value pairs for information about the alternative claim data to be presented through user interface elements on the interface. At least some of the user interface elements may be rich content to allow the patient to expand and input data for queries and other submissions to entities in the communication session. For example, when the patient accepts the alternative claims data, the service may generate a message (e.g., an e-fax) by populating the input into a template for the message, and the service or the patient (or other stakeholder on behalf of the patient) may send the message to the care provider to approve the substitute prescription.

In addition, the service may monitor for usage of the electronic card at a pharmacy service for a transaction to receive the substitute prescription at the value identified in the alternate claim data. When detected (e.g., use of the card at a position of sale (POS) device or an online transaction portal), the service may validate whether the card is used at the pharmacy service identified by the alternate claim data. If successfully validated, the service may identify a rule configured by the patient for the application of the value across accounts associated with the patient. The rule may specify a sequence to apply the value across the accounts and maximum for withdrawal from each account. With the identification, the service may select the accounts and apply the value to the accounts as specified by the rule. The service may determine a difference in the values between the original claim data and the alternate claim data to provide for presentation to the patient.

Furthermore, the service may maintain the distribution schedule for the claim assigned to the patient. The distribution schedule may define a series of time points by which corresponding target objectives are to be satisfied by the patient to transfer a specific amount to accounts of the patient. The distribution schedule may specify inputs for data to check against the target objectives. For example, the distribution schedule may specify that the patient is to refill the prescribed medication each month over the course of a year to receive the disbursement amount. The service may listen for data associated with the claim from the patient's device, pharmacy services, care provider services, or program provider services, among others. With the receipt of the data, the service may compare the data with the objectives of the time point. When the data satisfies the objectives, the service may send a request to a transactions processor to transfer the corresponding amount to the accounts of the patient.

In this manner, the database query service may provide for centralized management of claims data aggregated at the database to facilitate relatively easy access and query functions for alternative claims data for initial and/or ongoing data mining. The service may also facilitate for messaging in connection with the query across involved entities, including the patient, care provider, and the pharmacy, which otherwise would have lacked integrated communication channels. As a result, time and effort that would have been consumed in searching for the entirety of the claims data may now be drastically reduced. In addition, the database query service may decrease consumption of computing resources from redundant and duplicative storage of claims data and improve quality and integrity of claims data by specifying a standardized formatting for data maintained on the database. By providing the messaging interface, the service may increase the quality of human-computer interaction (HCl) in querying the database for claims data.

Aspects of the present disclosure are directed to systems, methods, and non-transitory computer readable media for querying databases for switching claims. A server may receive, from a client of a user, a first claim indicating (i) a prescription to be taken to address a condition, (ii) a first value for a receipt of the prescription, and (iii) a first pharmacy service of a plurality of pharmacy services through which the prescription is to be received. The server may obtain, for storage on a database, a plurality of second claims from the plurality of pharmacy services, each of the plurality of second claims indicating (i) the prescription to be taken, (ii) a respective second value for receipt of the prescription, and (iii) a corresponding second pharmacy service of the plurality of pharmacy services through which the prescription to be received. The server may identify, from the database, a second claim indicating the second value relative to the first value, from applying a recommendation engine on the first claim and the plurality of second claims. The server may detect an occurrence of a triggering condition associated with a likelihood of the user to accept switching of the first claim with the second claim. The server may transmit, responsive to detecting the occurrence of the triggering condition, a notification message to the client for presentation on a user interface to prompt the user to accept or reject the second claim instead of the first claim.

In one embodiment, the server may receive, from the client, an indication of an acceptance of the second claim via the user interface. The server may update, on the database, a user account associated with the user to switch assignment from the first claim to the second claim. In another embodiment, the server may transmit, responsive to the indication of the acceptance, a second notification message to the second pharmacy service to prompt for acceptance or rejection of the second claim. The server may receive, from the second pharmacy service, a second indication of acceptance of the second claim. The server may generate information for an electronic card to be used by the user to receive the prescription through the second pharmacy service at the second value.

In yet another embodiment, the server may receive, from the client, an indication of a rejection of the second claim via the user interface. The server may continue to monitor the database for a third claim from the plurality of second claims from the plurality of pharmacy services to switch the first claim, responsive to the indication of the rejection.

In yet another embodiment, the recommendation engine may identify, from the plurality of second claims, the second claim having one or more correspondences with the first claim. In yet another embodiment, the one or more correspondences includes at least one of a therapeutic correspondence or a generic correspondence between the prescription identified in the first claim and the prescription identified in the second claim.

In yet another embodiment, the recommendation engine may use a machine learning model to select the second claim from the plurality of second claims based on the first claim. In yet another embodiment, the machine learning model is trained according to a training dataset comprising a plurality of examples each identifying (i) a sample claim to be switched and (ii) a sample candidate claim to switch with the sample claim.

Aspects of the present disclosure are directed to systems, methods, and non-transitory computer readable media for dynamically updating chat message interfaces for database queries. A server may obtain for storage on a database, a plurality of claims from the plurality of pharmacy services. Each of the plurality of claims may indicate (i) the prescription to be taken, (ii) a respective value for receipt of the prescription, and (iii) a corresponding pharmacy service of the plurality of pharmacy services through which the prescription to be received. The server may identify, from the database, a claim indicating a first value relative to a second value initially assigned to a user, from executing a recommendation engine using the plurality of claims. The server may transmit a message object for presentation via a chat messaging interface on a client of the user, the message object identifying the claim for the prescription. The server may generate, responsive to an indication of acceptance of the claim by the user via the chat messaging interface, a digital document to indicate switch to the claim in accordance with an electronic facsimile format. The server may transmit the digital document to a care provider service via one or more communication channels including an electronic facsimile channel.

In one embodiment, the server may receive, from the care provider service, an indication of one of an approval or a rejection of the claim. The server may transmit, responsive to receipt of the indication, a notification message identifying the indication for presentation via the chat messaging interface of the client. In another embodiment, the server may transmit, responsive to receipt of the indication of an approval of the claim from a care provider service, a second message object to a pharmacy service identified in the claim. The server may receive an acknowledgment of receipt of the second claim from a pharmacy identified by the second claim.

In yet another embodiment, the server may initiate a chat session between the client and the pharmacy service to exchange messages via the chat messaging interface, responsive to the acknowledgement of receipt of the second claim. In yet another embodiment, the server may monitor, for a modification in the prescription assigned to the user via one or more data sources. The server may transmit, responsive to detecting the modification, a second message object for presentation via the chat messaging interface on the client of the user, the message identifying the modification in the prescription.

In yet another embodiment, the server may identify the message object from a message library comprising a plurality of message objects. Each of the plurality of message objects may define a respective plurality of user interface elements to be presented via the user interface. The server may generate the message object to indicate the claim for the prescription identified using the recommendation engine. In yet another embodiment, the chat message interface may present a plurality of user interface elements. At least one of the plurality of user interface elements may include a rich content field for input.

Aspects of the present disclosure are directed to systems, methods, and non-transitory computer readable media for linking data sources. A server of a benefits optimization computing network may receive, from a first pharmacy service, a transaction data indicating a transaction to receive a prescription by a user at a value using an electronic card associated with the benefits optimization platform. The server may select, from a plurality of claims on a database, a claim assigned to the user using the transaction data. The claim may indicate a second pharmacy service through which the prescription is received. The server may validate that the transaction is occurring at the first pharmacy service corresponding to a second pharmacy service identified in the claim. The server may identify, responsive to validating, a user account of the user defining a rule for applying transaction values to one or more accounts linked with the electronic card. The server may determine, for each of the one or more accounts, a respective transaction value in accordance with the rule. The server may transmit, to a transactions processor associated with each of the one or more accounts, a request to transfer the respective transaction value from a corresponding account.

In one embodiment, the server may determine a difference value based upon (i) the transaction value identified in the claim and (ii) a transaction value identified in a second claim previously assigned to the user. The server may update, on the database, a user account associated with the user with an indication of the different value to apply for subsequent claims of the user across the one or more accounts. In another embodiment, the server may transmit to a client associated with the user the difference value for presentation via the client.

In yet another embodiment, the server may determine to refrain from withdrawing from the one or more account linked to the electronic card of the user, responsive to failure to validate. In yet another embodiment, the rule defined in the user account identifies at least one of (i) a sequence in which to withdraw transaction values from the one or more accounts linked to the electronic card or (ii) a maximum value at which to withdraw from each of the one or more accounts.

In yet another embodiment, the electronic card may be issued by the benefits optimization platform to perform the transaction for the claim assigned to the user. In yet another embodiment, the server may receive, from a point-of-sale (POS) device associated with the pharmacy service, the transaction data including an identification of the pharmacy service.

Aspects of the present disclosure are directed to systems, methods, and non-transitory computer readable media of generating computer-readable instructions to switch data structures from databases. A server may identify, for a user associated with a user, a first data structure indicating (i) a access token to authorize intake associated with a condition and (ii) a first service of a plurality of services associated with the first data structure. The server may receive a plurality of second data structures for storage on a database. Each of the plurality of second data structures may indicate (i) the access token to authorize intake and (ii) a second service of the plurality of services associated with the access token to be received. The server may access the database to select a second data structure from the plurality of second data structures using the first data structure, the second data structure indicating a respective second value within a margin of a first value of the first data structure. The server may aggregate, on the database, data associated with the user, the first data structure. The server may execute a model using data associated with the user, the first data structure, and the second data structure to generate a computer-readable instruction identifying a value for the user to accept the second data structure instead of the first data structure. The server may transmit a notification message to the computing device for presentation on a user interface to prompt the user to accept or reject the computer-readable instruction to switch from the first data structure to the second data structure.

In one embodiment, the server may access the database to retrieve a subset of second data structures from the plurality of second data structures using first data structure, each of the subset of second data structures indicating a respective second value within a margin of a first value of the first data structure. The server may generate, for each of the subset of second data structures, a respective computer-readable instruction identifying a respective value to induce the user to accept a corresponding second data structure of the subset of second data structures. The server may transmit the notification identifying the subset of second data structures and the respective computer-readable instruction for each of the subset of second data structures.

In another embodiment, the model may include a machine learning (ML) model trained using a training dataset comprising a plurality of examples. Each of the plurality of examples may identify (i) a respective third data structure, (ii) a respective fourth data structure to switch with the third data structure, (ii) a respective computer-readable instruction to switch from the third data structure to the fourth data structure, and (iv) an indication of one of acceptance or rejection of the fourth data structure by a respective user. In yet another embodiment, the model may include a function of the data associated with the user, the first data structure, and the second data structure to generate the computer-readable instruction to induce the user to accept the second data structure.

In yet another embodiment, the server may receive, via the user interface from the computing device, an indication of acceptance of the computer-readable instruction. The server may update a user account associated with the user to switch assignment from the first data structure and to the second data structure and to include an identification of the computer-readable instruction. In yet another embodiment, the server may transmit, responsive to the indication of the acceptance, a second notification message to a computer-readable instruction provider service to prompt for acceptance or rejection of the computer-readable instruction. The server may receive, from the computer-readable instruction provider service, a second indication of acceptance of the computer-readable instruction to switch to the second data structure. The server may generate, in accordance with the computer-readable instruction, a distribution schedule to transfer the value to one or more accounts associated with the user. In yet another embodiment, the server may receive, via the user interface from the computing device, an indication of a rejection of the second data structure. The server may continue to monitor the database for a third data structure from the plurality of second data structures to switch with the first data structure, responsive to the indication of the rejection.

In yet another embodiment, the server may receive, from at least one of the computing device or the first service, a query identifying the first data structure for which an alternative data structure is to be identified. In yet another embodiment, the server may determine a likelihood of acceptance of switching from the first data structure to the data structure. The server may transmit the notification message, responsive to an occurrence of a trigger condition corresponding to the likelihood satisfying a threshold. In yet another embodiment, the server may generate the computer-readable instruction comprising electronic card information associated with the second data structure.

Aspects of the present disclosure are directed to systems, methods, and non-transitory computer readable media for validating time points for data structures with access tokens in networked environments. A server may identify a data structure assigned to a user and a computer-readable instruction associated with the data structure, the data structure indicating (i) a access token to authorize intake associated with a condition and (ii) a service through which the access token is to be received. The server may obtain a distribution schedule defining a plurality of time points, at each of which a corresponding portion of a value is to be transferred to one or more accounts associated with the user in response to satisfying a corresponding target condition associated with the data structure in accordance with the computer-readable instruction. The server may receive, prior to a time point of the plurality of time points, data associated with the data structure from the user. The server may determine that the data satisfies the target condition defined by the distribution schedule for the time point. The server may transmit, to a transactions processor associated with the one or more accounts, a request to transfer the corresponding portion of the value of the time point to the one or more accounts, responsive to determining that the data satisfies the target condition.

In one embodiment, the server may receive, prior to a second time point of the plurality of time points, second data associated with the data structure from the user. The server may determine that the second data does not satisfy the target condition defined by the distribution schedule for the second time point. The server may refrain from transmitting a second request to transfer the corresponding portion of the value to the transaction server, responsive to determining that the second data does not satisfy the target condition.

In another embodiment, the server may transmit a notification message to the computing device for presentation on a user interface to indicate a transfer of the corresponding portion of the value to the one or more accounts associated with the user. In yet another embodiment, the server may update a user account associated with the user to identify a remaining portion of the value available to be transferred across the one or more accounts.

In yet another embodiment, the server may receive transaction data indicating a transaction to receive the access token by the user at a second value defined by the data structure. In yet another embodiment, the server may retrieve, from a computer-readable instruction provider service, the distribution schedule of the computer-readable instruction accepted by the user to accept the data structure. In yet another embodiment, the target condition may include at least one of: (i) a use of the access token by the user, (ii) a performance of an activity by the user, or (iii) an issuance of the access token by the service.

In yet another embodiment, the server may identify the data structure to which assignment for the user is switched from a second data structure previously associated with the user. In yet another embodiment, the server may obtain the distribution schedule of a computer-readable instruction associated with the data structure. In yet another embodiment, the server may receive historical data identifying activities performed by the user via the computing device.

It is to be understood that both the foregoing general description and the following detailed description are exemplary and explanatory and are intended to provide further explanation of the embodiments described herein.

### BRIEF DESCRIPTION OF THE DRAWINGS

The accompanying drawings constitute a part of this specification and illustrate embodiments of the subject matter disclosed herein.

FIG. 1 depicts a block diagram of a system for handling claims data on databases, in accordance with an illustrative embodiment;

FIG. 2 depicts a block diagram of a system for query claims data on databases, in accordance with an illustrative embodiment;

FIG. 3 depicts a block diagram of a process for finding alternative claims by querying on databases, in accordance with an illustrative embodiment;

FIG. 4 depicts a flow diagram of a method of query claims data on databases, in accordance with an illustrative embodiment;

FIG. 5 depicts a block diagram of a system for messaging information for querying claims databases, in accordance with an illustrative embodiment;

FIG. 6A depicts a screenshot of a messaging interface to present alternative claims on a client in the system for messaging information, in accordance with an illustrative embodiment;

FIG. 6B depicts a screenshot of a messaging interface to present a chat session on a client in the system for messaging information, in accordance with an illustrative embodiment;

FIG. 7 depicts a block diagram of a method of messaging information for querying claims databases, in accordance with an illustrative embodiment;

FIG. 8 depicts a block diagram of a system for applying configurations for electronic cards, in accordance with an illustrative embodiment;

FIG. 9 depicts a block diagram of an architecture for processing transactions using electronic cards, in accordance with an illustrative embodiment;

FIG. 10 depicts a flow diagram of a method of applying configurations for electronic cards, in accordance with an illustrative embodiment;

FIG. 11 depicts a block diagram of a system for generating programs to switch claims from databases, in accordance with an illustrative embodiment;

FIG. 12 depicts a flow diagram of a method of generating programs to switch claims from databases, in accordance with an illustrative embodiment;

FIG. 13 depicts a block diagram of a system for maintaining distribution schedules for claims, in accordance with an illustrative embodiment; and

FIG. 14 depicts a flow diagram of a method of maintaining distribution schedules for claims, in accordance with an illustrative embodiment.

### DETAILED DESCRIPTION

Reference will now be made to the illustrative embodiments illustrated in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one ordinarily skilled in the relevant art and having possession of this disclosure, are to be considered within the scope of the subject matter disclosed herein. The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

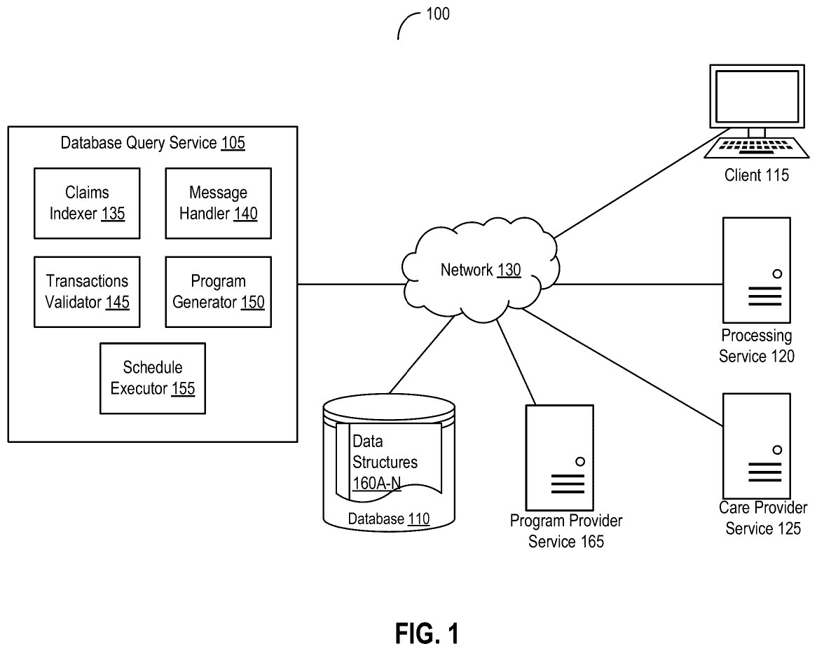

FIG. 1 depicts a block diagram of a system 100 for handling claims data on databases. In overview, the system 100 may include at least one database query service 105 (sometimes herein referred to generally as service or server), at least one database 110, at least one client 115 (sometimes herein generally referred to as a computing device), at least one pharmacy service 120 (sometimes herein generally referred to as a service), at least one care provider service 125, and at least one program provider service 165, among others, communicatively coupled with one another via at least one network 130. The database query service 105 may include at least one claims indexer 135, at least one message handler 140, at least one transactions validator 145, at least one program generator 150, and at least one schedule executor 155, among others. The database may store, maintain, otherwise include a set of data structures 160A-N (hereinafter generally referred to as data structures 160). Each of the components described herein may be implemented using hardware or a combination of hardware and software (e.g., one or more processors coupled with memory to execute computer-readable instructions) for performing functionalities such as those detailed herein.

Embodiments may comprise additional or alternative components or omit certain components from those of FIG. 1 and still fall within the scope of this disclosure. Various hardware and software components of one or more public or private networks 130 may interconnect the various components of the system 100. Non-limiting examples of such networks may include Local Area Network (LAN), Wireless Local Area Network (WLAN), Metropolitan Area Network (MAN), Wide Area Network (WAN), and the Internet. The communication over the network may be performed in accordance with various communication protocols, such as Transmission Control Protocol and Internet Protocol (TCP/IP), User Datagram Protocol (UDP), and IEEE communication protocols.

The data query service 105 may be any computing device including one or more processors coupled with memory with software capable of performing the various processes and tasks described herein. The data query service 105 may be associated with an entity managing the data structures 160 (sometimes herein referred to as a claim or claim data structure) on the database 110. Although shown as a single data query service 105, the data query service 105 may include any number of computing devices. The data query service 105 may be in communication with the client 115, the pharmacy service 120, and the care provider service 125 via the network 130. The data query service 105 may include several subsystems to perform the operations described herein. Executing on the data query service 105, the claims indexer 135 may maintain the data structures 160 on the database 110 and handle queries for data structures 160 in the database 110. The message handler 140 may facilitate communications among the client 115, the pharmacy service 120, and the care provider service 125 over the network 130 in relation to querying the data structures 160 on the database 110. The transactions validator 145 may manage use of electronic cards in connection with data structures 160 on the database 110. The program generator 150 may generate instructions to induce acceptance of substitute data structures 160. The schedule executor 155 may manage transfer in connection with the instructions when the substitute data structure 160 is accepted.

The client 115 may be any computing device including one or more processors coupled with memory with software capable of performing the various processes and tasks described herein. The client 115 may be operated by or associated with a patient (sometimes herein referred to as a user, a member, or a subject), a caretaker of the patient, or any entity associated with the patient, among others. The client 115 may be in communication with the data query service 105, the pharmacy service 120, and the care provider service 125 via the network 130. The client 115 may be used by the patient to access the functionalities of the data query service 105. The client 115 may access the data query service 105 via an application (e.g., native application installed thereon or a web application accessible through a web browser).

The pharmacy service 120 may be any computing device including one or more processors coupled with memory with software capable of performing the various processes and tasks described herein. The pharmacy service 120 may be operated by or associated with a pharmacy entity, a physical location of a pharmacy, or a pharmacy supplier, among others. The client 115 may be in communication with the data query service 105, the client 115, and the care provider service 125 via the network 130. The pharmacy service 120 may generate and provide data structures 160 to store and maintain on the database 110 based on available prescriptions. The entity associated with the pharmacy service 120 may provide the prescription identified in the data structure 160 assigned to the patient.

The care provider service 125 may be any computing device including one or more processors coupled with memory with software capable of performing the various processes and tasks described herein. The care provider service 125 may be operated by or associated with a care provider, such as a clinician, a doctor, a dentist, a nurse, a physical therapist, a psychologist, or a pathologist, among others. The care provider service 125 may be in communication with the data query service 105, the client 115, and the pharmacy service 120, via the network 130. The care provider service 125 may approve the prescription identified in the data structures 160 assigned to the patient associated with the client 115.

The program provider service 165 may be any computing device including one or more processors coupled with memory with software capable of performing the various processes and tasks described herein. The program provider service 165 may be operated by or associated with a program provider, such as a pharmacy benefit manager (PBM) or any third-party entity separate from the pharmacy service 120 or the care provider service 125, among others. The program provider service 165 may be in communication with the data query service 105, the client 115, and the pharmacy service 120, via the network 130. The program provider service 165 may manage or provide programs to switch to alternate claim data. In some embodiments, the program provider service 165 may be part of the pharmacy service 120 or the care provider service 125.

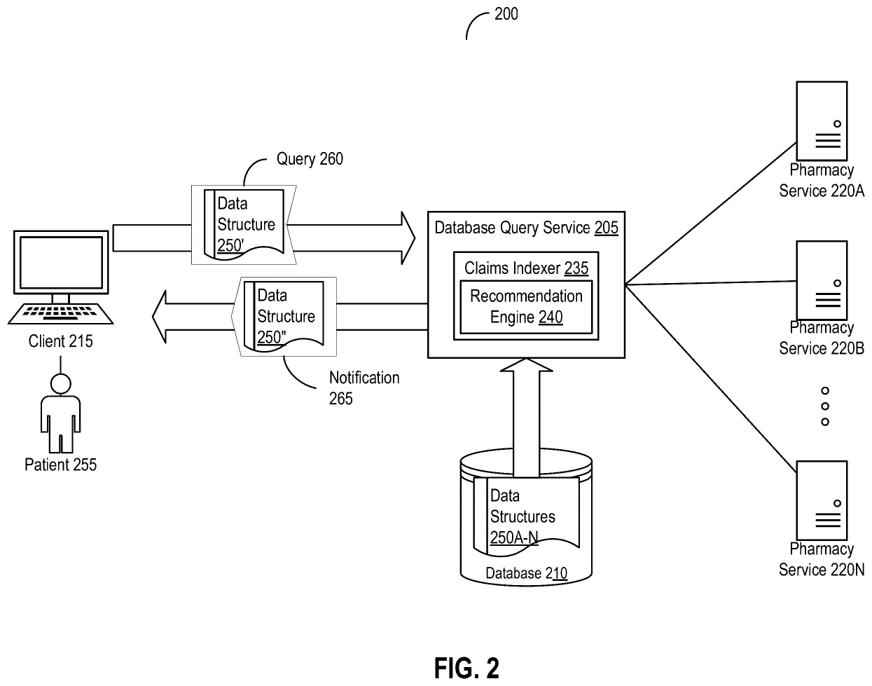

FIG. 2 depicts a block diagram of a system 200 for query claims data on databases. In overview, the system 200 may include at least one database query service 205 including at least one claims indexer 235 including at least one recommendation engine 240, at least one database 210 storing and maintaining a set of data structures 250A-N (hereinafter generally referred to as data structures 250), at least one client 215 operated by or associated with at least one patient 255, and one or more pharmacy services 220A-N (hereinafter generally referred to as pharmacy service 220), among others. Embodiments may comprise additional or alternative components or omit certain components from those of FIG. 2 and still fall within the scope of this disclosure. Various hardware and software components of one or more public or private networks may interconnect the various components of the system 200. Each component in system 200 (such as the database query service 205, the client 215, and the pharmacy service 220) may be any computing device comprising one or more processors coupled with memory and software, and capable of performing the various processes and tasks described herein.

As used herein, a data structure 250 refers to data containing various types of data fields for initiating execution of a transaction associated with a claim. Each data structure 250 may correspond to a prescription drug available for provision through the pharmacy service 220. In some embodiments, each data structure 250 may include an access token (e.g., generated by the pharmacy service 220 or a care provider service) to authorize intake of the prescription drug by the patient 255. The drug for a prescription (as indicated by the data structure 250 data) may be used to address a medical condition, alleviate symptoms associated with such medical conditions, or prevent disease or illness, among others. The prescription data (in the data structure 250 data) may be defined in terms of, for example, a type of medication, dosage, frequency, administration route, and duration, among others. Each data structure 250 may include, identify, or indicate, for example, a corresponding prescription available for provision or distribution taken through the corresponding pharmacy service 220, a value (e.g., amount billed and any adjustments) associated with receipt of the prescription, and the pharmacy service 220 through which the prescription is to be provided, among others. The access token may include a set of alphanumeric characters identifying the prescribed drug and prescription data, among other data. In some embodiments, the data structure 250 can identify or indicate whether the data structure 250 itself (along with the prescription, the pharmacy service 220, and the value) is to be processed (e.g., in accordance with benefits structure, negotiated pricing, or discount to value) by a third-party entity, such as a health insurance entity or a benefits entity, among others. The data structures 250 may be maintained as one or more machine-readable files in accordance with a standardized data format (e.g., extensible markup language (XML) or JavaScript Object Notation (JSON)) across different pharmacy services 220. In some embodiments, each data structure 250 may include a set of field-value pairs. The field-value pairs may include information as detailed herein.

The data structures indexer 235 executing on the database query service 205 store and maintains the set of data structures 250 on the database 210. The data structures indexer 235 may gather, collect, or otherwise aggregate the set of data structures 250 from one or more pharmacy services 220. In some embodiments, the data structures indexer 235 may use data structures 250 from drug manufacturers and care providers to maintain the set of data structures 250.

In conjunction, the client 215 may provide, transmit, or send at least one query 260 for the patient 255 to the database query service 205. The query 260 may include or identify at least one data structure 250′ for which an alternative or a substitute is to be identified. The data structure 250′ of the query 260 may be generated or populated by the patient 255 using a user interface presented via the application on the client 215. In some embodiments, another entity besides the client 215 may send the query 260. For instance, a care provider service, an insurance firm, or the pharmacy service 220 may send the query 260 on behalf of the patient 255. The data structure 250′ may include, identify, or indicate: a prescription initially assigned to be taken by the patient 255 to address a condition, a value (e.g., amount billed and any adjustments) associated with receipt of the prescription, and a pharmacy service 220 through which the patient 255 is to receive the prescription, among others.

The claims indexer 235 retrieves, identifies, or otherwise receives the query 260 for the patient 255. Upon receipt, the claims indexer 235 may parse the data structure 250′ to extract or identify: the prescription initially assigned to be taken by the patient 255, the value associated with receipt of the prescription, and the pharmacy service 220 through which the patient 255, among others. In some embodiments, the data structure 250′ of the query 260 can identify or indicate whether the data structure 250′ itself was processed by the third-party entity. The claims indexer 235 may identify additional information associated with the patient 255 or the data structure 250′ through other data sources. In some embodiments, the claims indexer 235 may store and maintain the data structure 250′ on the database 210.

With the receipt, the claims indexer 235 searches, identifies, or otherwise identifies one or more candidate claims from the set of data structures 250 from the database 210. The candidate data structures may correspond to a subset of data structures 250 available for switching with data structures from patients, including the data structure 250′ for the patient 255 identified in the query 260. Upon identification, the claims indexer 235 identifies or selects at least one data structure 250″ from the one or more candidate data structures to assign or provide to the patient 255. The selection of the data structure 250″ may be based on the data structure 250′ identified in the query 260 (e.g., the prescription, the value, the identification of the pharmacy service 220, and an indication of whether the data structure is processed by the third-party entity) and the additional information associated with the patient 255 or the data structure 250′ through other data sources. The selected data structure 250″ may identify a value lower than the value identified in the data structure 250′ of the query 260.

To select at least one data structure 250″ from the candidate data structures to assign to the patient 255, the claims indexer 235 may maintain a set of rules. The set of rules may be defined, maintained, or otherwise configured on the recommendation engine 240. At least one rule may be to select based on a therapeutic correspondence between the prescription in the candidate data structure and the prescription identified in the data structure 250′ of the query 260. In accordance with the rule on therapeutic correspondence, the data structures indexer 235 may select the data structure 250″ from the candidate data structures having a therapeutic correspondence with the data structure 250′ of the query 260. For example, the prescription in the data structure 250″ and the prescription identified in the data structure 250′ may both address the same medical condition. In some embodiments, the data structures indexer 235 may call or invoke the recommendation engine 240 to select the data structure 250″ from the candidate data structures having the therapeutic correspondence according to the set of rules configured on the recommendation engine 240.

Continuing on, at least another rule may be to select based on a generic correspondence between the prescription in the candidate data structure and the prescription identified in the data structure 250′ of the query 260. In accordance with the rule on generic correspondence, the data structures indexer 235 may select the data structure 250″ from the candidate data structures having a generic correspondence with the data structure 250′ of the query 260. For instance, the prescription in the data structure 250″ may be genericized version of the prescription identified in the data structure 250′. The data structure 250″ selected using the rules may identify a value lower than the initially assigned value identified in the data structure 250′ of the query 260. In some embodiments, the claims indexer 235 may call or invoke the recommendation engine 240 to select the data structure 250″ from the candidate data structures having the generic correspondence according to the set of rules configured on the recommendation engine 240.

In some embodiments, the claims indexer 235 may initiate, train, or otherwise establish at least one machine learning (ML) model to select at least one data structure 250″ from the candidate data structures to assign to the patient 255. The ML model may be of any architecture, such as regression model (e.g., linear or logistic), a decision tree, a random forest, a support vector machine (SVM), artificial neural network (ANN), a Naïve Bayes classifier, or a clustering algorithm, among others. The ML model may be configured or maintained on the recommendation engine 240. The ML model may be established or trained using a training dataset in accordance with supervised (e.g., for ANN) or unsupervised learning (e.g., for clustering algorithm). The training dataset may identify a number of examples. Each example may identify a sample data structure to be switched with another data structure and a set of sample candidate data structures to switch with the sample data structure. Both data structures may include information in a similar format as the data structures 250. With the establishment, the claims indexer 235 may apply the data structure 250′ of the query 260 into the ML model to output the selected model. In some embodiments, the claims indexer 235 may call or invoke the recommendation engine 240 to select the data structure 250″ from the candidate data structures using the ML model configured on the recommendation engine 240.

The claims indexer 235 may monitor for an occurrence of at least one triggering condition. The triggering condition may be associated with a likelihood of the user accept the switching from the initial data structure 250′ to the substitute data structure 250″. To monitor, the claims indexer 235 may aggregate, obtain, or otherwise retrieve data indicative of the user's acceptance of the switching from the initial data structure 250′ to the substitute data structure 250″. The data may identify or include, for example, the receipt of the query 260, a time to refill the prescription for the drug, and geolocation data on the patient 255 (e.g., relative to the pharmacy service 220 identified in the data structure 250″), among others. Based on the data, the claims indexer 235 may calculate, generate, or otherwise determine the predicted likelihood that the user will accept the switch from the initial data structure 250 to the data structure 250″. With the determination, the claims indexer 235 may compare the likelihood to a threshold. The threshold may delineate, specify, or otherwise define a value for the likelihood at which the trigger condition is determined to have occurred. If the likelihood satisfies (e.g., is greater than or equal to) the threshold, the claims indexer 235 may detect the occurrence of the triggering condition. On the other hand, if the likelihood does not satisfy (e.g., less than) the threshold, the claims indexer 235 may continue monitor for the occurrence of the triggering condition.

The claims indexer 235 returns, send, or otherwise transmits at least one notification message 265 to the client 215 (or a computing device associated with another entity). In some embodiments, the claims indexer 235 may transmit the notification message 265 to the client 215 responsive to detecting the triggering condition. The notification message 265 may include or identify the data structure 250″ selected to switch the data structure 250′ initially assigned to the patient 255 as identified in the query 260. The data structure 250″ may be provided for an electronic card (e.g., a virtual card) to be used by the patient 255 to receive the prescription from the pharmacy service 220 at the value as identified in the data structure 250″. The notification message 265 may be presented on a user interface on the client 215 to prompt the patient 255 (or user) to accept or reject the data structure 250″ instead of the initial data structure 250′. Upon receipt, the client 215 may display, render, or otherwise present information based on the notification message 265.

With the transmission of the notification message 265, the claims indexer 235 may wait for an indication of acceptance or rejection of the data structure 250″ identified in the notification message 265 by the patient 255. When the indication of rejection is received from the client 215, the claims indexer 235 may continue to monitor the database 210 for new data structures to switch from the initial data structure 250′. When the indication of the acceptance is received, the claims indexer 235 may update an account (or a user profile) associated with the patient 255 to switch assignment of the patient 255 from the original data structure 250′ to the new data structures 250″.

In some embodiments, upon acceptance by the client 215, the claims indexer 235 may also transmit another notification message to the pharmacy service 220 identified in the data structure 250″ to prompt for acceptance or rejection of the data structure 250″. If the indication of rejection is received from the pharmacy service 220, the claims indexer 235 may forward or provide the indication of the rejection to the client 215. The claims indexer 235 also may continue to monitor the database 210 for new data structures to switch from the initial data structure 250′. In contrast, when the indication of the acceptance is received, the data structures indexer 235 may forward or provide the indication of the acceptance to the client 215. In addition, the claims indexer 235 may update account associated with the patient 255 to switch assignment of the patient 255 from the original data structure 250′ to the new data structures 250″.

In some embodiments, the claims indexer 235 may initially provide or transmit the data structure 250″ for the electronic card, upon acceptance of the data structure 250 by the patient 255, the pharmacy service 220 (e.g., identified in the data structure 250″), or a care provider associated with the patient 255, among others. In some embodiments, the claims indexer 235 may generate the information for the electronic card to use for the data structure 250″, upon identifying or detecting prior use of electronic cards by the patient 255. The information for the electronic card may include or identify the data structure 250″ itself and information about the patient 255 (e.g., including insurance and account data), among others. In some embodiments, the client 215 may receive the electronic card information along with the data structure 250″ from the database query service 205.

By performing the above functionalities, the database query service 205 can carry out a values-based rewards function. Under the values-based rewards function, the database query service 205 can handle the query 260 submitted by the patient 255 to search for substitute data structures 250″ for the initially assigned data structure 250′. Based on the information associated with the patient 255, the database query service 205 can identify a substitute data structure 250″ to provide to the patient 255 to select for switching. Once switched from the initial data structure 250′ to the new data structure 250″, the patient 255 may be provided with a reward from selecting a lower value to use the pharmacy service 220 as identified in the substitute data structure 250″. In this manner, the values-based rewards function carried by the database query service 205 can incentivize patients 255 to make use of the system.

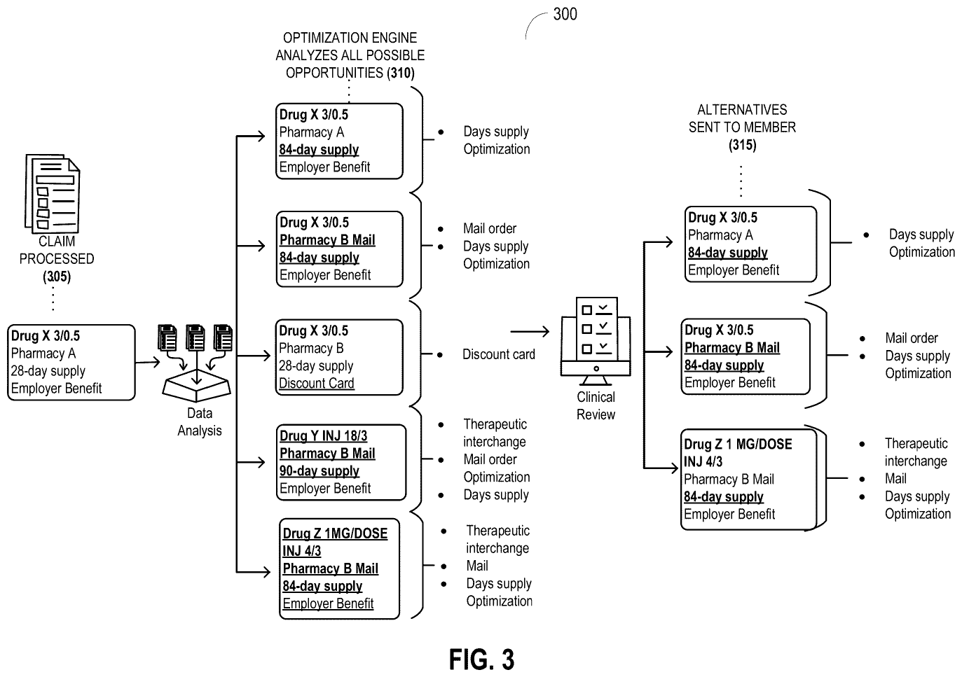

FIG. 3 depicts a block diagram of a process 300 for finding alternative claims by querying on databases. The process 300 may be implemented or performed using any of the components detailed herein. Embodiments may include additional, fewer, or different operations from those described in the process 300. The process 300 may be performed by a server executing machine-readable software code, though it should be appreciated that the various operations may be performed by one or more computing devices and/or processors.

At step 305, a service may process claims submitted by a patient. In the depicted example, the service may receive the claims data identifying a drug “X” to be received from pharmacy “A” for 28 days and to be paid through an employer benefit. This claims data may be initially assigned to the patient. Upon receipt, the service may process the claims data to conform to a standardized format.

At step 310, the service may use an optimization engine to analyze candidate or possible claims data to replace the claim data originally assigned to the patient. The candidate claims data may be aggregated from various pharmacy services or drug manufacturers, and the candidate claims data may be maintained on a database. The selection of the alternate claims data may be based on an optimization of the day supply, the value for receipt of the prescription, and therapeutic or generic correspondence between the prescriptions. In accordance with the optimization, the service may select one of the candidate claims data as an alternate claims data to provide to the patient.

At step 315, the service may provide the alternative claims data to the patient. In providing, the service may first perform a clinical evaluation of the prescription in the alternative claim data. For example, the service may transmit the alternative claims data for approval by a care provider associated with the patient. Upon approval, the service may provide the one or more alternative claims to the patient.

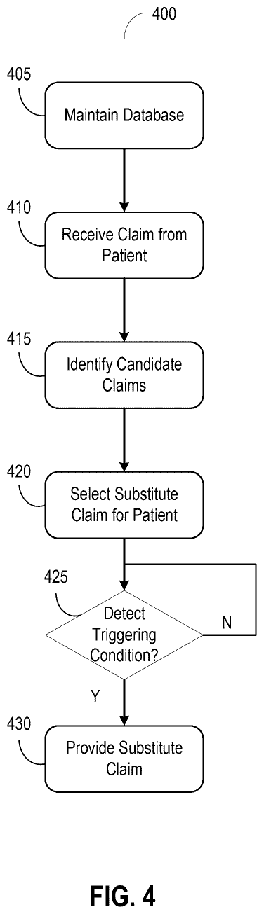

FIG. 4 depicts a flow diagram of a method 400 of query claims data on databases.

The method 400 may be implemented or performed using any of the components detailed herein. Embodiments may include additional, fewer, or different operations from those described in the method 400. The method 400 may be performed by a server executing machine-readable software code, though it should be appreciated that the various operations may be performed by one or more computing devices and/or processors. Under the method 400, at step 405, a service may maintain a database of claims. At step 410, the service may receive a claim from a patient. At step 415, the service may identify one or more candidate claims for the patient. At step 420, the service may select at least one substitute claim for the patient. At step 425, the service may detect whether there is an occurrence of a triggering condition. When there is no detection, the service may continue to monitor for the triggering condition. At step 430, when the occurrence of the triggering condition is detected, the service may provide the substitute claim to the patient.

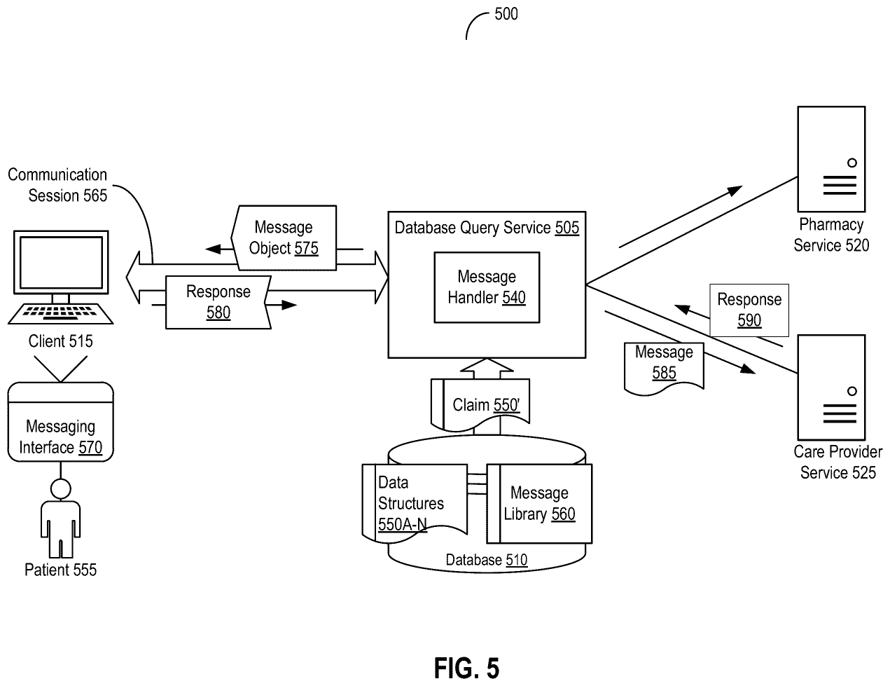

FIG. 5 depicts a block diagram of a system 500 for messaging information for querying claims databases. In overview, the system 500 may include at least one database query service 505 including at least one message handler 540, at least one database 510 storing and maintaining a set of data structures 550A-N (hereinafter generally referred to as data structures 550) and a message library 560, at least one client 515 operated by or associated with at least one patient 555, at least one pharmacy service 520, and at least one care provider service 525, among others. Embodiments may comprise additional or alternative components or omit certain components from those of FIG. 5 and still fall within the scope of this disclosure. Various hardware and software components of one or more public or private networks may interconnect the various components of the system 500. Each component in system 500 (such as the database query service 505, the database 510, the client 515, the pharmacy service 520, and the care provider service 525) may be any computing device comprising one or more processors coupled with memory and software, and capable of performing the various processes and tasks described herein.

The message handler 540 executing on the database query service 505 may initiate and establish at least one communication session 565 with the client 515 (or a computing device of another associated entity). The communication session 565 may facilitate or may be used to present at least one messaging interface 570 on the client 515 for querying the database 510 for data structure 550. For example, the patient 555 may use the messaging interface 570 to generate queries to send via the communication session 565 to search the database 510 for alternate data structure 550′. The communication session 565 may provide for secure exchange of messages (e.g., a chat messaging interface) between the patient 555 on the client 515 and any one or more other entities, such as the pharmacy service 520, the care provider service 525, or an insurance firm, among others.

In conjunction, the message handler 540 retrieves, receives, or identifies a response to a query to switch from a data structure 550 originally assigned to the patient 555 to at least one alternate data structure 550′. The data structure 550′ may be identified by a recommendation engine applied on the data structures 550 of the database 510. In some embodiments, the message handler 540 may listen, wait, or otherwise monitor for the response to the query to switch the data structures. The query may be from the patient 555 or another entity, such as the care provider or the insurer associated with the patient 555. Upon identification, the message handler 540 may parse the alternate data structure 550′ to extract or identify: the prescription available to be taken by the patient 555, a value associated with receipt of the prescription, and the pharmacy service 520 through which the prescription is to be provided, among others.

With the identification, the message handler 540 may create, write, or generate at least one message object 575 using the data structure 550′. The message object 575 may identify, specify, or otherwise define a set of field-value pairs to be presented via one or more user interface elements on the messaging interface 570. Each field-value pair may include a field identifying a type of information and a value identifying corresponding data assigned to the field. At least a portion of the field-value pairs may be generated from the information in the data structure 550′. For instance, the message handler 540 may populate the values in the field-value pairs with the prescription information, the value, and the identification of the pharmacy service 520 as identified in the data structure 550′. At least one of the user interface elements for presentation on the messaging interface 570 may accept, obtain, or otherwise receive user input when presented.

In some embodiments, the message handler 540 may identify or select the message object 575 to use to provide for the patient 555. The message handler 540 may store and maintain the message library 560 on the database 510. The message library 560 may include a set of available message objects from which to select to provide over the communication session 565 for presentation on the messaging interface 570. Each message object may identify, specify, or otherwise define a set of field-value pairs to be presented via one or more user interface elements on the messaging interface 570. In some embodiments, each message object may be associated with a corresponding trigger to select the corresponding message object for provision. In some embodiments, the message handler 540 may select or identify the message object 575 in response to detection of the trigger associated with the patient 555. For example, the trigger may include a detection of the response to a query of the database 510 or a modification in the prescription for the patient 555.

With the generation, the message handler 540 provides, send, or otherwise transmits the message object 575 via the communication session 565 for presentation of the set of field-value pairs on the one or more user interface elements of the messaging interface 570. At least one of the user interface elements identified in the message object 575 may be used to indicate one of an acceptance or rejection of the data structure 550′ for the patient 555. The user interface elements may include, for example, command buttons, text field, check boxes, radio buttons, sliders, icons, tabs, progress bars, sliders, among others to present the set of field-value pairs on the messaging interface 570. At least one of the user interface elements may include a rich content field (e.g., an image, icon to a file, an interactive graphic, or multimedia content). The rich content field may present and receive rich content on the messaging interface 570. The user interface elements may be embedded into an area within the messaging interface 570. For example, the user interface elements used to present the field-value pairs of the data structures 550′ may be within a defined area of a web application accessed through the client 515.

Upon receipt, the client 515 may listen or monitor for at least one user interaction with the user interface elements rendered, displayed, or otherwise presented on the messaging interface 570. The interaction may be with the user interface element to indicate acceptance or rejection of the data structure 550′ for the patient 555. The user interface elements for the field-value pairs for the data structure 550′ may be presented, rendered, or displayed adjacent to the user interface element for accepting or rejecting the data structure 550′. Upon detection of the interaction, the client 515 may generate at least one response 580 (sometime referred herein as an input) to identify or indicate the acceptance or the rejection of the data structure 550′. When the interaction is to indicate acceptance, the client 515 may generate the response 580 to indicate the acceptance of the data structure 550′. When the interaction is to indicate rejection, the client 515 may generate the response 580 to indicate the rejection of the data structure 550′. With the generation, the client 515 may return, send, or otherwise transmit the response 580 to the database query service 505 over the communication session 565.

The message handler 540 retrieves, identifies, or receives the response 580 from the client 515 over the communication session 565. With receipt, the message handler 540 may process or parse the response 580 to determine whether the indication is the acceptance or the rejection of the data structure 550′. If the indication is to reject the data structure 550′, the message handler 540 may determine to refrain from assigning the alternate data structure 550′ to the patient 555. The message handler 540 may continue to monitor for responses to queries to switch from the initially assigned data structure 550 to one or more alternate data structures 550′ from the database 510. On the other hand, if the indication is to accept, the message handler 540 may assign the alternate data structure 550′ to the patient 555.

When the response 580 includes the indication to accept the data structure 550′, the message handler 540 may create, write, or otherwise generate at least one notification message 585 to be provided to the care provider service 525. The notification message 585 may be generated in accordance with a template using the data structure 550′. The template may define an arrangement of the information (e.g., field-value pairs for the data structure 550′) for presentation on the notification message 585. For example, the template may specify a sequence and placement of the field-value pairs for the data structure 550′ in an electronic fax message. Upon generation, the message handler 540 may provide, send, or otherwise transmit the notification message 585 to the care provider service 525.

The care provider service 525 retrieves, identifies, or otherwise receives the notification message 585 from the database query service 505. Upon receipt and presentation of the notification message 585, the care provider service 525 may listen or monitor for at least one user interaction. The interaction may be with the user interface element to indicate approval or denial of the data structure 550′ by a care provider associated with the patient 555. Upon detection of the interaction, the care provider service 525 may generate at least one response 590 (sometimes referred to as an input) to identify or indicate the approval or the denial of the data structure 550′. When the interaction is to indicate approval, the care provider service 525 may generate the response 590 to indicate the approval of the data structure 550′. When the interaction is to indicate denial, the care provider service 525 may generate the response 590 to indicate the denial of the data structure 550′. With the generation, the care provider service 525 may return, send, or otherwise transmit the response 590 to the database query service 505.

The message handler 540 may in turn retrieve, identify, or receive the response 590 from the care provider service 525. In some embodiments, the message handler 540 may store and maintain the response 590 on the database 510. With receipt, the message handler 540 may process or parse the response 590 to determine whether the indication is the approval or the denial of the data structure 550′. If the indication is to deny the data structure 550′, the message handler 540 may determine to refrain from assigning the alternate data structure 550′ to the patient 555. The message handler 540 may continue to monitor for responses to queries to switch from the initially assigned data structure 550 to one or more alternate data structures 550′ from the database 510. On the other hand, if the indication is to accept, the message handler 540 may determine to confirm assignment of the alternate data structure 550′ to the patient 555.

In some embodiments, the messaging handler 540 may extend the communication session 565 with the client 515 to other entities, such as the pharmacy service 520 or the care provider service 525. When the indication is approval, the message handler 540 may send, provide, or otherwise transmit an indication of the approval to the pharmacy service 520. The indication message may be generated in accordance with a template using the data structure 550′. The template may define an arrangement of the information (e.g., field-value pairs for the data structure 550′) for presentation on the indication message. Upon generation, the message handler 540 may provide, send, or otherwise transmit the indication message to the pharmacy service 520.

The pharmacy service 520 retrieves, identifies, or otherwise receives the indication message from the database query service 505. Upon receipt and presentation of the indication message, the pharmacy service 520 may listen or monitor for at least one user interaction. The interaction may be with the user interface element to indicate approval or denial of the data structure 550′ by a care provider associated with the patient 555. Upon detection of the interaction, the pharmacy service 520 may generate at least one response to indicate acknowledgment. With the generation, the pharmacy service 520 may return, send, or otherwise transmit the response to the database query service 505.

Upon receipt of the acknowledgment, the messaging handler 540 may extend the communication session 565 from the client 515 to the pharmacy service 520. With the extension, the communication session 565 may be used to exchange messages between the client 515 and the pharmacy service 520. The messages may be inputted and presented on the messaging interface 570 on the client 515. The pharmacy service 520 may also have an interface similar to the message interface 570 to enter and receive messages.

In some embodiments, the message handler 540 may monitor for a modification to the prescription identified in the data structure 550′ for the patient 555. The monitoring may be subsequent to approval from the care provider service 525 and acknowledgment by the pharmacy service 520. The message handler 540 may monitor any number of sources to detect the change, such as from the client 515, the insurance firm, the care provider service 525, or the pharmacy service 520, among others. Upon detection, the message handler 540 may create, write, or otherwise generate a message object for the modification. The generation of the message object may be based on the prescription identified in the modification and at least a portion of the data structure 550′. The message object may identify or include a set of field-value pairs to be presented as one or more user interface on the messaging interface 570. Upon generation, the message handler 540 may send, provide, or otherwise transmit the message object via the communication session 565 for presentation on the messaging interface 570.

The message handler 540 may present other message objects 575 for presentation via the messaging interface 570. In some embodiments, the message handler 540 may identify a set of data structures 550 assigned to the patient 555 (e.g., initially assigned data structures 550 or subsequently assigned alternate data structures 550′) from the database 510. Each of the data structures 550 may identify at least one respective prescription to be taken by the patient 555. With the identification, the message handler 540 may send, provide, or otherwise transmit an identification of the set of data structures 550 via the communication session 565. The set of data structures 550 may be provided or presented as a set of corresponding user interface elements on the messaging interface 570. Each user interface element may identify a corresponding thread of messages within the communication session 565. For instance, each user interface element may allow for pop-up or expansion to display the text of the messages associated with a corresponding data structure 550.

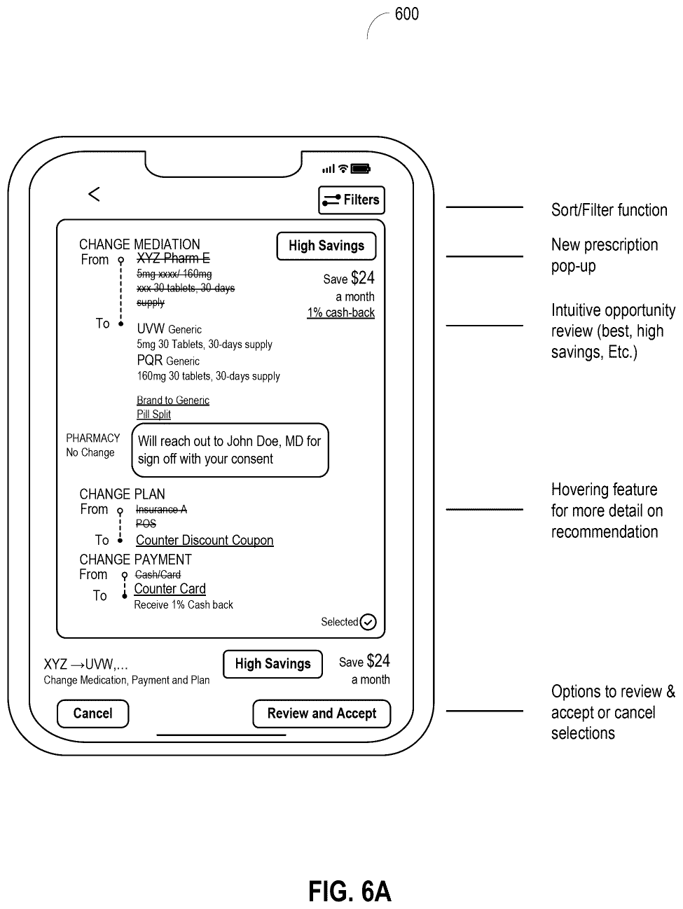

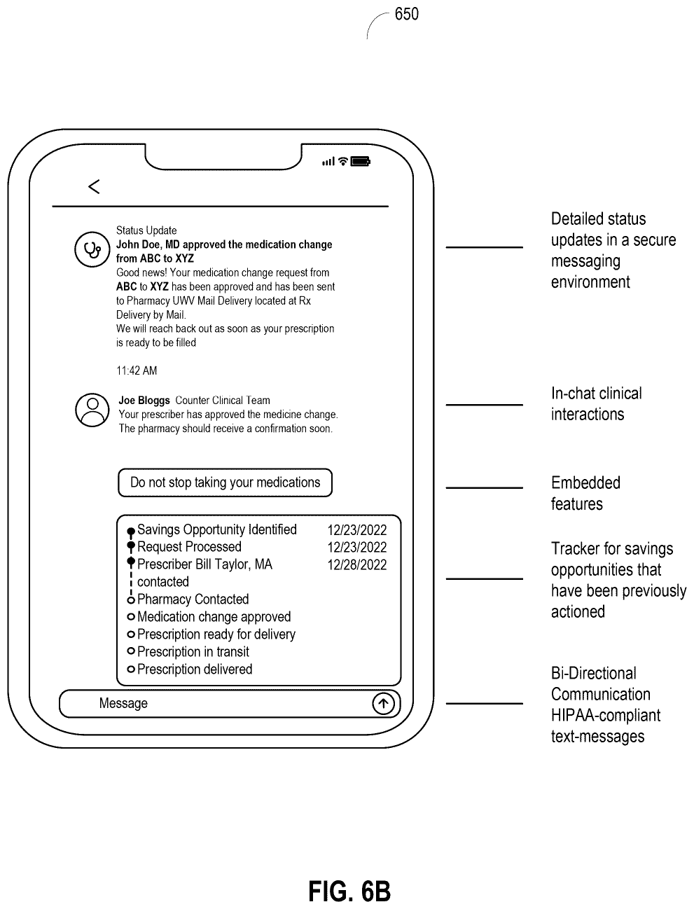

FIG. 6A depicts a screenshot of a messaging interface 600 to present alternative claims on a client in the system 500 for messaging information. As depicted, the messaging interface 600 may provide a sort or filter function, a new prescription pop-up element, an overview of the claim, an element to access additional information, and an option to accept or reject the selections. FIG. 6B depicts a screenshot of a messaging interface 650 to present a chat session on a client in the system 500 for messaging information. As depicted, the messaging interface 650 may provide a detailed status update (e.g., from the care provider) and a chat interactivity between the patient and the pharmacy. The messaging interface 650 may also provide for tracking of savings opportunities and bi-direction communication with other entities.

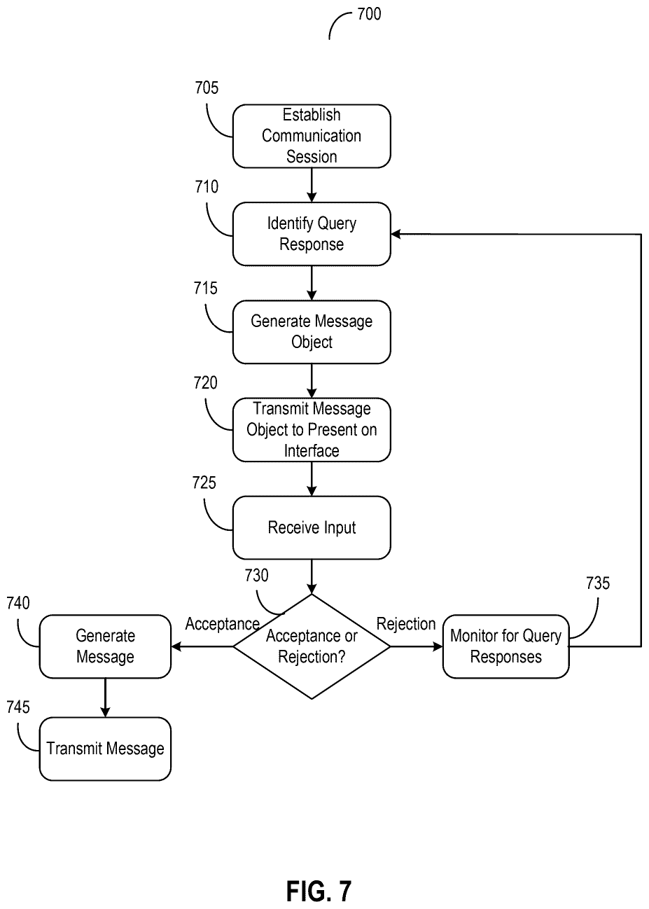

FIG. 7 depicts a block diagram of a method 700 of messaging information for querying claims databases. The method 700 may be implemented or performed using any of the components detailed herein. Embodiments may include additional, fewer, or different operations from those described in the method 700. The method 700 may be performed by a server executing machine-readable software code, though it should be appreciated that the various operations may be performed by one or more computing devices and/or processors. Under the method 700, at step 705, a service may establish a communication session. At step 710, the service may identify a query response. At step 715, the service may generate a message object. At step 720, the service may transmit the message object to present via an interface. At step 725, the service may receive an input via the interface. At step 730, the service may determine whether acceptance or rejection of a claim in the query response. At step 735, if the input indicates rejection, the service may monitor for query responses and may repeat the functionality from step 710. At step 740, the service may generate a message to provide to a care provider service. At step 745, the service may transmit the message to the care provider service.

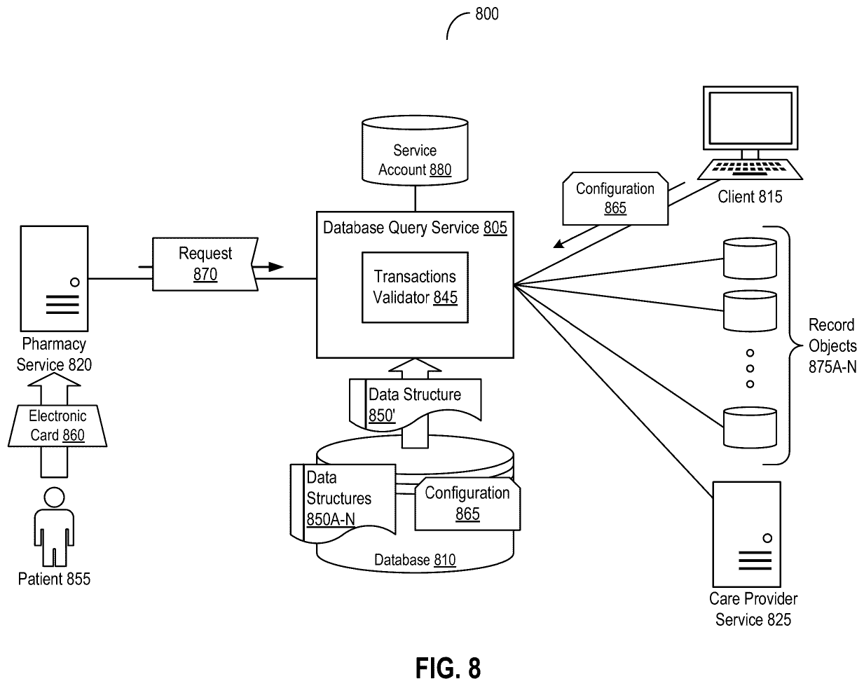

FIG. 8 depicts a block diagram of a system 800 for linking data sources. In overview, the system 800 may include at least one database query service 805 including at least one transactions validator 845, at least one database 810 storing a set of data structures 850A-N (hereinafter generally referred to as data structures 850), at least one client 815, at least one pharmacy service 820, and at least one care provider service 825, among others. Embodiments may comprise additional or alternative components or omit certain components from those of FIG. 8 and still fall within the scope of this disclosure. Various hardware and software components of one or more public or private networks may interconnect the various components of the system 800. Each component in system 800 (such as the database query service 805, the database 810, the client 815, the pharmacy service 820, and the care provider service 825) may be any computing device comprising one or more processors coupled with memory and software, and capable of performing the various processes and tasks described herein.

The transactions validator 845 executing on the database query service 805 retrieves, identifies, or otherwise receives at least one configuration 865 from the client 815 associated with the patient 855. The database query service 805 may be of a benefits optimization computing network handling data structures and processing transactions associated with the prescriptions for patients. In some embodiments, the transactions validator 845 may receive the configuration 865 from a computer device of another entity on behalf of the patient 855. The configuration 865 may specify, identify, or otherwise define at least one rule for selecting one or more record objects 875A-N (hereinafter generally referred to as record objects 875, and sometimes referred herein as patient account) to which to apply values of data structures 850. Each record object 875 may be a financial account maintained by a financial institution (e.g., a bank or brokerage) on behalf of the patient 855 and may identify a balance value against which a value of the data structures 850 are to be applied (e.g., added or deducted). The record objects 875 may include, for example, a checking account, a savings account, a flexible spending account (FSA), a health savings account (HSA), a medical savings account (MSA), an employer's health reimbursement arrangement (HRA) account, or a prescription discount card, among others.

Upon receipt, the transactions validator 845 may process or parse the configuration 865 to extract or identify the rule. The rule of the configuration 865 may define a sequence of record objects 875 to which to apply the values of the data structures 850. The rule may also define a respective maximum amount for each record object 875 that can be applied to the values of the data structures 850. The rule of the configuration 865 may identify which of the record objects 875 to apply the values of the data structures 850. With the identification, the transactions validator 845 may store the configuration 865 (or the rule) on the database 810. In some embodiments, the transactions validator 845 may store and maintain an association between the configuration 865 and at least one user account of the patient 855 on the database 810. The account may identify one or more record objects 875 associated with the patient 855.

In conjunction, the transactions validator 845 retrieves, receives, or identifies a response to a query to switch from a data structure 850 originally assigned to the patient 855 to at least one alternate data structure 850′. In some embodiments, the transactions validator 845 may listen, wait, or otherwise monitor for the response to the query to switch the data structures. The query may be from the patient 855 or another entity, such as the care provider or the insurer associated with the patient 855. Upon identification, the transactions validator 845 may parse the alternate data structure 850′ to extract or identify: the prescription available to be taken by the patient 855, a value associated with receipt of the prescription, and the pharmacy service 820 through which the prescription is to be provided, among others. Separately, the alternate data structure 850′ may be used to generate and issue at least one electronic card 860 for the patient 855 to obtain the specified prescription at the value through a designated pharmacy service 820. The electronic card 860 may be associated with the benefits optimization platform and may be linked with the record objects 875 associated with the patient 855. The electronics card 860 may be issued by the benefits optimization platform to perform the transaction for the data structures (e.g., the data structure 850′) assigned to the patient 855.

In addition, the transactions validator 845 listens, waits, or otherwise monitors for a use of the electronic card 860 through the pharmacy service 820 (e.g., at a physical storefront or an online merchandise portal). The electronic card 860 may be a physical or virtual card associated with the patient 855 and may include information transacting for obtaining the specified prescription at the value through a designated pharmacy service 820 in accordance with the data structure 850′. The transactions validator 845 may monitor for the use of the electronic card 860 via a point-of-sale (POS) device (e.g., a counter register device, a table POS system, a card and chip reader, or a touch screen PoS device) associated with the pharmacy service 820. For example, the transaction validator 845 may be in communication with the POS device located in the physical storefront for the pharmacy service 820 to listen for events, such as the scanning of the electronic card 860.

In monitoring, the transactions validator 845 may retrieve, receive, or otherwise identify an indication of a request 870 for transaction at the pharmacy service 820. As the electronic card 860 may identify the database query service 805, the request 870 may be routed from the pharmacy service 820 to the database query service 805. The request 870 may include transactions data indicating a transaction to receive the prescription by the patient 855 using the electronics card 860 from the pharmacy service 820. The transactions data of the request 870 identify the value to be applied as identified by the data structure 850′ and by extension the electronic card 860. In some embodiments, the transactions validator 845 may receive the request 870 from the POS device associated with the pharmacy service 820. When the request 870 is received, the transactions validator 845 may detect or identify the request as the use of the electronic card 860.

Upon detection of the use, the transactions validator 845 may validate the use of the electronic card 860 at the pharmacy service 820. To validate, the transactions validator 845 may identify or select the data structure 850′ assigned to the patient 855. From the data structure 850′, the transactions validator 845 may identify the pharmacy service 820 through which the prescription is to be received. With the identification, the transactions validator 845 may determine whether the pharmacy service 820 at which the use is detected corresponds (e.g., matches) the pharmacy service 820 identified in the data structure 850′. When the pharmacy services 820 differ, the transactions validator 845 may determine that the use of the electronic card 860 is invalid. The transactions validator 845 may transmit an indication of invalid use to the client 815 associated with the patient 855 or the pharmacy service 820 at which the use is detected. In addition, the transactions validator 845 may refrain from withdrawing from any of the record objects 875 linked to the electronic card 860. On the other hand, when the pharmacy services 820 correspond, the transactions validator 845 may determine that the use of the electronic card 860 is valid. The transactions validator 845 may determine to apply the value to any of the record objects 875 and may continue processing the transaction request.

With the validation, the transactions validator 845 select or identify a user account of the patient 855 defining the configuration 865. From the account, the transaction validator 845 may select or identify one or more record objects 875 associated with the patient 855 to which to apply the value. From the overall set of record objects 875, the transactions validator 845 may select a subset of record objects 875 in accordance with the rule of the configuration 865. For example, the transactions validator 845 may select the checking account and the FSA account associated with the patient 855 from the overall set of record objects 875. In some embodiments, the transactions validator 845 may identify the rule of the configuration 865 based on the information associated with the electronic card 860. Using the identified rule, the transactions validator 845 may select one or more from the overall set of record objects 875.

Upon selection, the transactions validator 845 may calculate, generate, or otherwise determine a respective value to apply for each of the one or more record objects 875. The determination of the value to apply may be in accordance with the rule defined in the configuration 865. For instance, the transactions validator 845 may determine the value from the savings account up to the specified maximum and then withdraw the remaining value from the HSA associated with the patient 855. Using the determined value, the transactions validator 845 applies the value across the selected record objects 875. To apply, the transactions validator 845 may provide, send, or otherwise transmit a request to transfer the determined transaction value to a respective transactions processor associated with each of the record objects 875. The request may identify the value to be subtracted, deducted, or otherwise withdrawn from the one or more record objects 875 according to the heuristics, operational configurations, or rules.

In some embodiments, the transactions validator 845 may apply the value to at least one service account 880 associated with the database query service 805, while waiting acknowledgment (e.g., of withdrawal or transfer) from the transactions validator associated with the record objects 875. The service account 880 may be a financial account maintained by a financial institution (e.g., a bank or brokerage) on behalf of the entity associated with the database query service 805. The value may be applied (e.g., withdrawn or deducted) to the service account 880 for a defined period of time (e.g., 1 day to 2 weeks), while waiting for the confirmation of transfer from the record objects 875. Upon receipt of the acknowledgement, the transactions validator 845 may apply a reimbursement value (e.g., by adding or depositing) to the service account 880.

With the application, the transactions validator 845 calculates, generates, or otherwise determines a difference value based on the value identified in the originally assigned data structure 850 and the value identified in the alternate data structure 850′. The difference value may represent an amount in savings arising from switching from the original data structure 850 to the alternate data structure 850′. The transactions validator 845 may determine the difference value, in response to application of the value across the one or more record objects 875 upon detection of the use of the electronic card 860. Upon determination, the transactions validator 845 may store the difference value on the database 810 for use in subsequent applications of values in other data structures 850 of the patient 855. In some embodiments, the transactions validator 845 may update the account associated with the patient 855 with the indication of the difference value to apply for subsequent data structures across the record objects 875. The transactions validator 845 may also transmit, provide, or send an indication of the difference value to the client 815 associated with the patient 855. The presentation of the indication may increase the likelihood that the patient 855 associated with the client 815 will accept future switches of data structures 850.

The transactions validator 845 transmits, provides, or sends one or more messages in connection with the application of the value to various entities, such as the client 815 associated with the patient 855, the pharmacy service 820, and the care provider service 825, among others. In some embodiments, the transactions validator 845 may provide, send, or transmit a message to present an indication of the application of the value across the one or more selected record objects 875. The message may be presented via an interface of the client 815, and may be, for example, a notification that the value has been successfully withdrawn from the identified record objects 875.

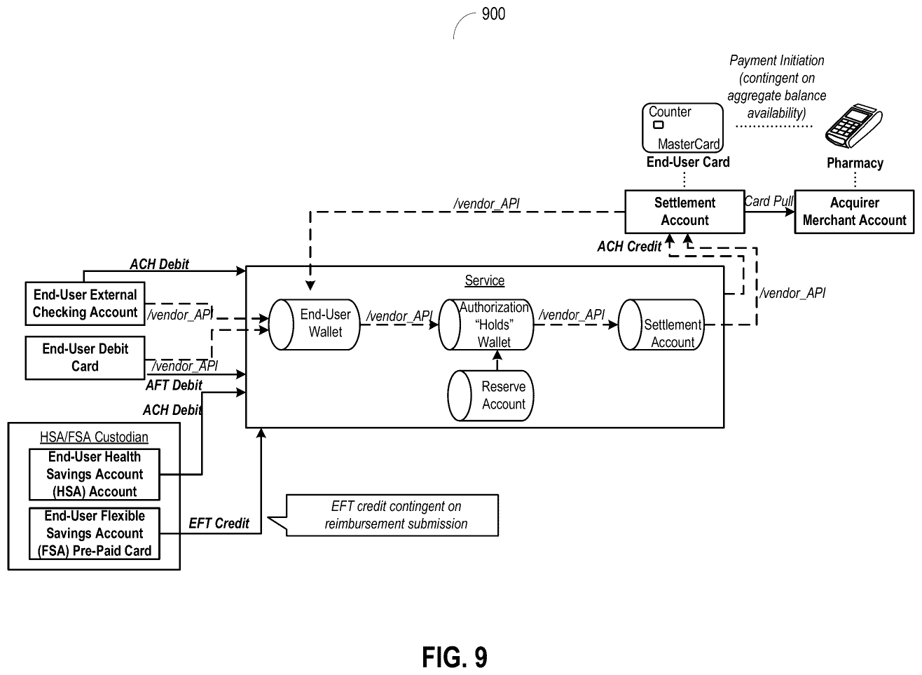

FIG. 9 depicts a block diagram of an architecture 900 for processing transactions using electronic cards. In the architecture 900, the service may detect the use of an end-user card at a pharmacy to obtain a prescription in accordance with a data structure assigned to the patient. When used the pharmacy, a point-of-sale (POS) device may transfer the value identified in the end-user card from a settlement account associated with the service to a merchant account. The settlement account may perform an application programming interface (API) call with the service to access related accounts. Upon detection, the service may use one or more API calls to transfer values from accounts associated with a patient, such as an external checking account, a debit card, a health savings account (HSA), or a flexible savings account (FSA), among others. The transfer may be in accordance with various electronic funds transfer (EFT) protocols, such as an automated clearing house (ACH) or automated funds transfer (AFT), among others. The service may pull the values from the accounts to transfer to an end-user wallet account, and in turn to an authorization wallet. The service may use balance on a reserve account to apply (e.g., add) to the authorization wallet. The service may transfer the value from the authorization wallet to the settlement account, and in turn apply the value to the settlement account for the end-user card.

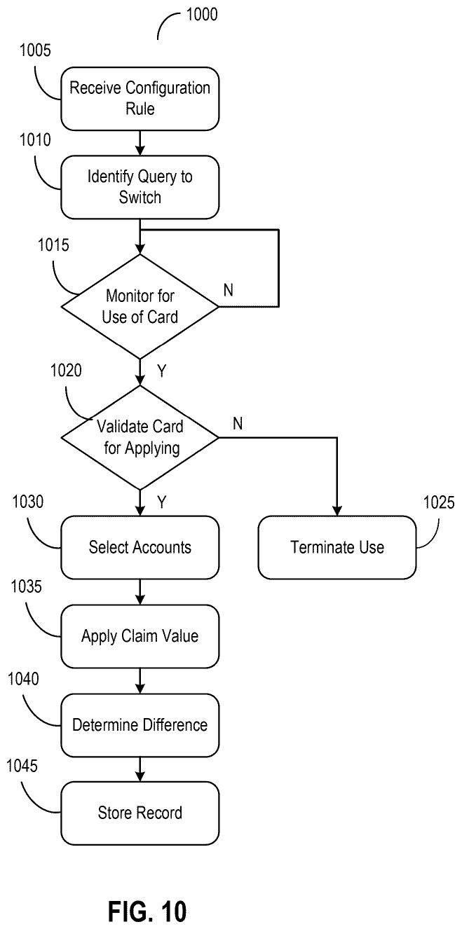

FIG. 10 depicts a flow diagram of a method 1000 of applying configurations for electronic cards. The method 1000 may be implemented or performed using any of the components detailed herein. Embodiments may include additional, fewer, or different operations from those described in the method 1000. The method 1000 may be performed by a server executing machine-readable software code, though it should be appreciated that the various operations may be performed by one or more computing devices and/or processors. Under the method 1000, at step 1005, a service may receive a configuration rule. At step 1010, the service may identify a query to switch. At step 1015, the service may monitor for a use of an electronic card. At step 1020, when the use is detected, the service may determine whether the card is validated for application. At step 1025, if validation is not successful, the service may terminate the transaction. Otherwise, at step 1030, if the validation is successful, the service may select accounts. At step 1035, the service may apply claim value. At step 1040, the service may determine a different. At step 1045, the service may store a record.

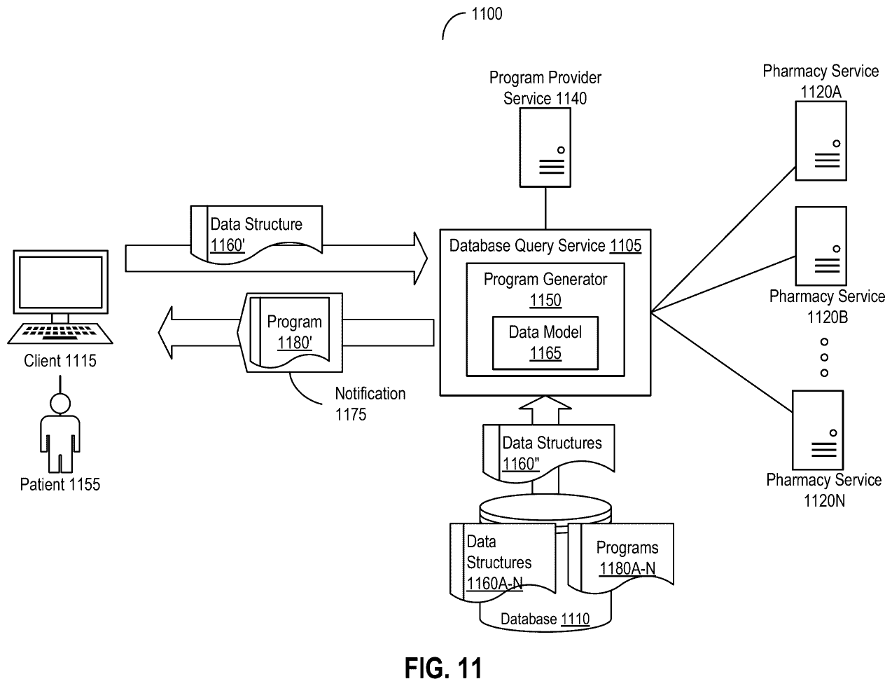

FIG. 11 depicts a block diagram of a system 1100 for generating programs to switch claims from databases. In overview, the system 1100 may include at least one database query service 1105 including at least one program generator 1150, at least one database 1110, at least one client 1115, one or more pharmacy services 1120A-N (hereinafter generally referred to as pharmacy services 1120), and at least one program provider service 1140, among others. The database 1110 may store and maintain a set of data structures 1160A-N (hereinafter generally referred to as data structures 1160) and a set of programs 1180A-N (hereinafter generally referred to as programs 1180). The program generator 1150 may include at least one data model 1165.

Embodiments may comprise additional or alternative components or omit certain components from those of FIG. 11 and still fall within the scope of this disclosure. Various hardware and software components of one or more public or private networks may interconnect the various components of the system 1100. Each component in system 1100 (such as the database query service 1105, the database 1110, the client 1115, the pharmacy service 1120, and the program provider service 1140) may be any computing device comprising one or more processors coupled with memory and software, and capable of performing the various processes and tasks described herein.

In further detail, the program generator 1150 executing on the database query service 1105 retrieves, selects, or otherwise identifies at least one data structure 1160′ for the patient 1155. The data structure 1160′ may identify or indicate a prescription initially assigned to be taken by the patient 255 to address a condition, a value (e.g., amount billed and any adjustments) associated with receipt of the prescription, and a pharmacy service 220 through which the patient 255 is to receive the prescription, among others. The data structure 1160′ may be initially assigned to the patient 1155 and may be one which an alternative or substitute data structure is to be identified. In some embodiments, the program generator 1150 may retrieve, identify, or otherwise receive the data structure 1160′ as part of a query for the patient 255 to the database query service 1105. In some embodiments, the program generator 1150 may retrieve or identify the data structure 1160′ as assigned to the patient 1155 from the database 1110.

The program generator 1150 searches, identifies, or otherwise identifies one or more candidate data structures from the set of data structures 1160 by accessing the database 1110. The candidate data structures may correspond to a subset of data structures 1160 available for switching with data structures from patients, including the data structure 1160′ for the patient 1155. The selection of the candidate data structures may be in accordance with a set of rules or a machine learning model as detailed herein. In some embodiments, the program generator 1150 may identify or select candidate data structures 1160″ from the set of data structures 1160, with values within a margin of the value of the originally assigned data structure 1160′. The margin may define or identify an upper and lower limit to a deviation from the value of the originally assigned data structure 1160′. For example, the program generator 1150 may select candidate data structures 1160″ with prescriptions to address the same condition as the original data structure 1160′ and values that are within 5-10% of the value of the original data structure 1160′ as specified by the margin.

The program generator 1150 may maintain and store a set of programs 1180 on the database 1110, which may be tailored for particular patients 1155. As used herein, the program 1180 may refer to a data structure containing various types of data fields for facilitating switching of assignment of data structures 1160. The program 1180 may be or include computer readable or executable instructions, such as an application (e.g., a native application or mobile application), an applet (e.g., a web-based or Java-based applet), a plug-in (or an add-on or extension), or a smart contract of a distributed ledger (e.g., blockchain), among others. Each program 1180 may be tailored and used to induce or guide the patient 1155 to switch from the originally assigned data structure 1160′ to one of the candidate data structures 1160. The program 1180 may define, specify, or otherwise identify a value (e.g., an incentive amount, reward credit, or other types of rewards) to transfer to the accounts of the patient 1155 upon switching from the originally assigned data structure 1160′ to the candidate data structure. The program 1180 can identify or indicate whether the data structure 250 itself (along with the prescription, the pharmacy service 220, and the value) is to be processed (e.g., in accordance with benefits structure, negotiated pricing, or discount to value) by a third-party entity, such as a health insurance entity or a benefits entity, among others. At least one of the programs 1180 may be defined or provided by the pharmacy service 1120 or the program provider service 1140. At least one of the programs 1180 may be generated on the database query service 1105. The program 1180 may be maintained as one or more machine-readable files in accordance with an executable file format. In some embodiments, the program 1180 may include or identify distribution schedule for the value. The distribution schedule may define or identify a set of time points associated with different objectives.

In conjunction, the program generator 1150 collects, stores, or otherwise aggregates data associated with the patient 1155, the originally assigned data structure 1160′, and one or more candidate data structures 1160″, among other data mining or processing operations. In this way, the program generator 1150 (or other hardware and software components) initially and/or continually executes data mining operations for gathering and analyzing the various types of data associated with the patient 1155. The data may identify historical data about the patient 1155, such as activities by the patient 1350 on the client 1315, receiving prescriptions, refilling prescriptions, visits to a clinic associated with the pharmacy service 1120 or the care provider service 1125, and adherence to a prescription, among others. The data may identify or include information from the originally assigned data structure 1160′, such as the condition to be addressed, the prescription, the identification of the pharmacy service 1120, and the value, among others. The data may include information from the corresponding candidate data structure 1160″, such as the condition to be addressed, the prescription, the identification of the pharmacy service 1120, and the value, among others. The data may also include information from the pharmacy service 1120, such as fulfillment of prescriptions. The data may include information from the program provider service 1140, such as provision of values (e.g., incentive values) to other patients and prescriptions for which values were provided, among others.

The program generator 1150 applies the data model 1165 to data associated with the user, the originally assigned data structure 1160′, and one or more candidate data structures 1160″, among others. From applying the data model 1165, the program generator 1150 may generate at least one program 1180′ for the patient 1155. In some embodiments, the program generator 1150 may select the program 1180′ from the database 1110. In some embodiments, for each selected candidate data structure 1160″, the program generator 1150 may apply the data model 1165 to data associated with the user, the originally assigned data structure 1160′, and the corresponding candidate data structure 1160″. In general, the data model 1165 may be a heuristic model generated and updated using data associated with the user, the originally assigned data structure 1160′, and one or more candidate data structures 1160″, among others. From applying the data to the data model 1165, the program generator 1150 produces, outputs, or otherwise generates the program 1180′ for the corresponding candidate data structure 1160″. In some embodiments, the program generator 1150 may generate the program 1180′ for each candidate data structure 1160″.

The data model 1165 may be any function with inputs corresponding to the data and at least one output corresponding to the program 1180′ for the patient 1155 to switch from the originally assigned data structure 1160 to the corresponding candidate data structure 1160″. The heuristic function may include or define a mapping between information of the input data with one of the programs 1180 (e.g., maintained on the database 1110). For example, the function may be a weighted summation of enumerated values corresponding to the information in the input data to output another value corresponding to one of the programs 1180. The function may be used to generate output values to induce the patient 1155 to switch from the originally assigned data structure 1160′ to the corresponding candidate data structure 1160″. For instance, the output value may correspond to an optimal amount in incentives to push the patient 1155 to select the candidate data structure 1160″ over the originally assigned data structure 1160′. The function for the data model 1165 may be formed using heuristics gathered from previously aggregated data about the user, the originally assigned data structure 1160′, and one or more candidate data structures 1160″, among others. The function may be periodically updated using the data (e.g., over rolling time windows).

In some embodiments, the data model 1165 may include a machine learning (ML) model to generate the program 1180′. The ML model may be of any architecture, such as regression model (e.g., linear or logistic), a decision tree, a random forest, a support vector machine (SVM), artificial neural network (ANN), a Naïve Bayes classifier, or a clustering algorithm, among others. The ML model may be established or trained using a training dataset in accordance with supervised (e.g., for ANN) or unsupervised learning (e.g., for clustering algorithm). The training dataset may identify a set of examples. Each example may include or identify: input data (e.g., information about a sample patient, an originally assigned data structure for the sample patient and an alternate data structure); a program defining a value to switch the patient from the originally assigned data structure to the alternate data structure; and an indication of an acceptance or rejection of the alternate data structure by the sample patient, among others. The data structures may be of the same format as the data structures 1160. Using the examples, the data model 1165 may be trained to output or generate programs 1180 with values dependent on the originally assigned data structure 1160′ and the candidate data structure 1160″. The values may be set an optimal amount to induce the patient 1155 to select the candidate data structure 1160″ instead of the originally assigned data structure 1160, within the constraints set by other entities, such as the pharmacy service 1120 or the program provider service 1140.

In some embodiments, the program generator 1150 may listen for or monitor for an occurrence of at least one triggering condition. The triggering condition may be associated with a likelihood of the user accept the switching from the initial data structure 1160′ to the substitute data structure 1160″. To monitor, the program generator 1150 may aggregate, obtain, or otherwise retrieve data indicative of the user's acceptance of the switching from the initial data structure 1160′ to the substitute data structure 1160″. The data may identify or include, for example, the receipt of the query, a time to refill the prescription for the drug, and geolocation data on the patient 1155 (e.g., relative to the pharmacy service 11110 identified in the data structure 1160″), among others. Based on the data, the program generator 1150 may calculate, generate, or otherwise determine the predicted likelihood that the user will accept the switch from the initial data structure 1160 to the candidate data structure 1160″. With the determination, the program generator 1150 may compare the likelihood to a threshold. The threshold may delineate, specify, or otherwise define a value for the likelihood at which the trigger condition is determined to have occurred. If the likelihood satisfies (e.g., is greater than or equal to) the threshold, the program generator 1150 may detect the occurrence of the triggering condition. On the other hand, if the likelihood does not satisfy (e.g., less than) the threshold, the program generator 1150 may continue monitor for the occurrence of the triggering condition.

The program generator 1150 provides, sends, or otherwise transmits at least one notification message 1175 to the client 1115 (or a computing device associated with another entity). The notification message 1175 may be for presentation on a user interface to prompt the patient 1155 to accept or reject the program 1180′. In some embodiments, the program generator 1150 may transmit the notification message 1175 in response to detecting the occurrence of the triggering condition. The notification message 1175 may identify the program 1180′ including the value to provide to the patient 1155 to switch from the initially assigned data structure 1160′ to the candidate data structure 1160″. In some embodiments, the notification message 1175 may identify the program 1180′ for each of the candidate data structures 1160″. The notification message 1175 may include or identify information about each program 1180′, such as an identification of the pharmacy service 1120 of the candidate data structure 1160″ and the program provider service 1140 associated with the program 1180′. Upon receipt of the notification message 1175, the client 1115 may display, render, or otherwise present information based on the notification message 1175.

With the transmission of the notification message 1175, the program generator 1150 may wait for an indication of acceptance or rejection of the program 1180′ identified in the notification message 1175 by the patient 1155. When the indication of rejection is received from the client 1115, the program generator 1150 may continue to monitor the database 1110 for new data structures to switch from the initial data structure 1160′. When the indication of the acceptance is received, the program generator 1150 may update a user profile or an account associated with the patient 1155 to switch assignment of the patient 1155 from the original data structure 1160′ to the new data structures 1160″. The program 1180′ may also be downloaded and installed on the client 1115 as an executable (e.g., an application, an applet, a plug-in, or a self-executing smart contract). In addition, the program generator 1150 may issue, provide, or otherwise generate an electronic card to facilitate the receipt of the prescription by the patient 1155. The information for the electronic card may include or identify the data structure 1160″ itself and information about the patient 255 (e.g., including insurance and account data), among others.

In some embodiments, when the patient 1155 accept the program 1180′, the program generator 1150 provides, sends, or otherwise transmits another notification message to the pharmacy service 1120 identified in the alternate data structure 1160″ to prompt for acceptance or rejection of the data structure 1160″. If the indication of rejection is received from the pharmacy service 1120, the program generator 1150 may forward or provide the indication of the rejection to the client 1115. The program generator 1150 also may continue to monitor the database 210 for new data structures to switch from the initial data structure 1160′. In contrast, when the indication of the acceptance is received, the program generator 1150 may forward or provide the indication of the acceptance to the client 1115. In addition, the program generator 1150 may update the user profile associated with the patient 1155 to confirm the switching assignment from the original data structure 1160′ to the new data structure 1160″. In some embodiments, the program generator 1150 may write, output, or otherwise generate the distribution schedule for the program 1180′ to transfer the specified value to one or more accounts associated with the patient 1155.

In this manner, the database query service 1105 may provide an incentive program to enable patients 1155 to accept alternate data structures 1160″ different from the originally assigned data structure 1160′. The database query service 1105 can leverage or use the data model 1165 to select or generate programs 1180′ (also referred herein as computer-readable instructions) to induce or facilitate the assignment from the originally assigned data structure 1160′ to the alternate data structure 1160″, with the provision of a value set at an optimal amount to induce the patient 1155 to accept the new data structure 1160″. If the patient 1155 chooses to accept the program 1180′, the database query service 1105 may manage the execution of the program 1180′ in a seamless manner to facilitate the switching. The data-based incentive program carried by the database query service 205 can incentivize patients 255 to make use of the system and receive the prescription from other pharmacy services 1120 specified in the new data structure 1160′.

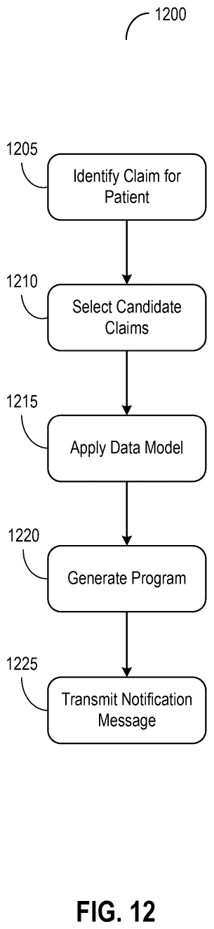

FIG. 12 depicts a flow diagram of a method 1200 of generating programs to switch claims from databases. The method 1200 may be implemented or performed using any of the components detailed herein. Embodiments may include additional, fewer, or different operations from those described in the method 1200. The method 1200 may be performed by a server executing machine-readable software code, though it should be appreciated that the various operations may be performed by one or more computing devices and/or processors. Under the method 1200, at step 1205, a server may identify a claim initially assigned to a patient. At step 1210, the server may select candidate claims for the patient. At step 1215, the server may apply a data model to information about the patient, the initially assigned claim, and each candidate claim. A step 1220, the server may generate a program from application of the data model. At step 1225, the server may transmit a notification message to prompt the patient to accept or reject the program to switch from the initially assigned claim to the candidate claim.

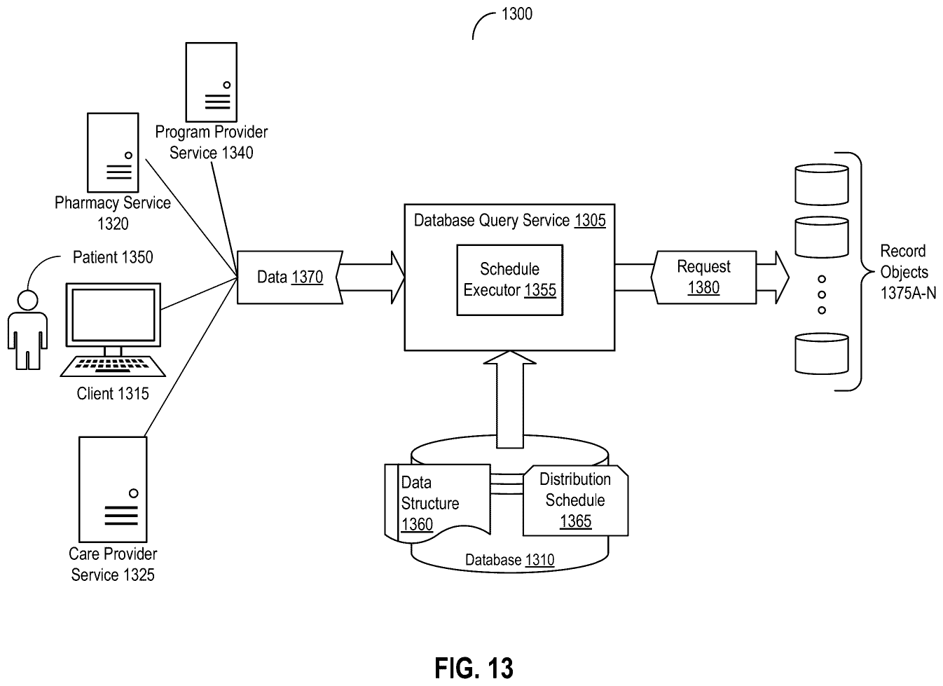

FIG. 13 depicts a block diagram of a system 1300 for maintaining distribution schedules for claims. In overview, the system 1300 may include at least one database query service 1305, at least one database 1310, at least one client 1315, at least one pharmacy service 1320, at least one care provider service 1325, and at least one program provider service 1340, among others. The database query service 1305 may include at least one schedule executor 1355. Embodiments may comprise additional or alternative components or omit certain components from those of FIG. 13 and still fall within the scope of this disclosure. Various hardware and software components of one or more public or private networks may interconnect the various components of the system 1300. Each component in system 1300 (such as the database query service 1305, the database 1310, the client 1315, the pharmacy service 1320, and the program provider service 1340) may be any computing device comprising one or more processors coupled with memory and software, and capable of performing the various processes and tasks described herein.

The schedule executor 1355 executing on the database query service 1305 obtains, receives, or otherwise identifies at least one data structure 1360 associated with a patient 1350. The data structure 1360 may identify or indicate a prescription assigned to be taken by the patient 1350 to address a condition, a value (e.g., amount billed and any adjustments) associated with receipt of the prescription, and a pharmacy service 1320 through which the patient 1350 is to receive the prescription, among others. The data structure 1360 may be one to which the patient 1350 switched to from an originally assigned data structure, in response to acceptance of a program defining the switching of assignments from the original data structure to the current data structure 1360. In some embodiments, the schedule executor 1355 may identify the data structure 1360 from a user profile associated with the patient 1350. The data structure 1360 and the user profile may be stored and maintained on the database 1310.

In addition, the schedule executor 1355 obtains, receives, or otherwise identifies at least one program associated with the data structure 1360. The program may be used to facilitate the assignment of the data structure 1360 to the patient 1350. In some embodiments, the program may have been used to induce the patient 1350 to switch from the originally assigned data structure to the current data structure 1360. For instance, the database query service 1305 may have transmitted a notification to the client 1315 associated with the patient 1350 to prompt the patient 1350 to accept the program. Upon acceptance, the database query service 1305 may switch the assignment to the current data structure 1360. The program may identify a value (e.g., a total amount for the incentive value or reward credit) to be transferred to the accounts of the patient 1350 in conjunction with the use of the assigned data structure 1360. In some embodiments, the schedule executor 1355 may identify the program associated with the data structure 1360 from the user profile associated with the patient 1350.

The schedule executor 1355 retrieves, identifies, or otherwise obtains at least one distribution schedule 1365 associated with the data structure 1360. The distribution schedule 1365 may be obtained from any number of sources. In some embodiments, the schedule executor 1355 may obtain or identify the distribution schedule 1365 from the program maintained on the database 1310 and associated with the data structure 1360. In some embodiments, the schedule executor 1355 may obtain or identify the distribution schedule 1365 from the pharmacy service 1320 associated with the data structure 1360 assigned to the patient 1350. In some embodiments, the schedule executor 1355 may obtain the distribution schedule 1365 from the program provider service 1340 associated with the program accepted by the patient 1350. In some embodiments, the schedule executor 1355 may obtain the distribution schedule 1365 from the client 1315 (e.g., from a mobile application interfacing with the database query service 1305).

The distribution schedule 1365 specifies, identifies, or otherwise defines a set of time points in accordance with the program used to assign the data structure 1360 to the patient 1350. For each time point, the distribution schedule 1365 may define a corresponding portion of the value (e.g., of the program accepted by the patient 1350) to be transferred to one or more record objects 1375A-N (hereinafter generally referred to as record objects 1375 and sometimes herein referred to as patient accounts) associated with the patient 1350. The distribution schedule 1365 may specify that the portion of the value is to be transferred in response to fulfilling or satisfying a target condition (e.g., an objective) associated with the data structure 1360 by the associated time point. For example, the distribution schedule 1365 may specify that 8.33% of the total amount is to be dispersed to the record objects 1375 each month across twelve months, each time the patient 1350 receives the specified prescription from the pharmacy service 1320 associated with the data structure 1360. The target condition may specify, identify, or otherwise include any number of objectives that the patient 1350 is to fulfill within the respective time point of the distribution schedule 1365, for example: a receipt of the prescription (e.g., identified in the data structure 1360), a performance an activity by the patient 1350, a detection of a visit at a location associated with the care provider service 1325, an issuance of the prescription by the pharmacy service 1320, or provision of a distribution notice from the program provider service 1340, among others. In some embodiments, the distribution schedule 1365 may define the set of time points to transfer the corresponding portions, independent of any target conditions.

In some embodiments, the schedule executor 1355 may write, produce, or otherwise generate the distribution schedule 1365 for the data structure 1360. In some embodiments, the schedule executor 1355 may generate the distribution schedule 1365 in accordance with a template. The template may identify or include the set of time points, a corresponding amount of the value (e.g., incentive or reward) to be transferred, and the target conditions, among others. The schedule executor 1355 may obtain or identify the data (e.g., timestamps, numerical values, and conditions) to include into the template from the program associated with the data structure 1360 assigned to the patient 1350. With the identification, the schedule executor 1355 may insert or include the values into the set of timepoints, the corresponding amounts, and the target conditions to form the distribution schedule 1365. The distribution schedule 1365 may be stored and maintained as one or more machine-readable files in accordance with a standardized data format (e.g., extensible markup language (XML) or JavaScript Object Notation (JSON)).

In conjunction, the schedule executor 1355 may wait for, listen for, or otherwise monitor data 1370 associated with the data structure 1360 assigned to the patient 1350. The schedule executor 1355 retrieves, identifies, or otherwise receives data 1370, relative to (e.g., before or concurrently) at least one of the time points defined by the distribution schedule 1365. The retrieval of the data 1370 may be to validate or check the time points of the distribution schedule 1365 to determine whether the corresponding target condition is satisfied. The data 1370 may identify or include any information associated with the patient 1350, the data structure 1360 assigned to the patient 1350, or the program accepted by the patient 1350, among others. The data 1370 may in general be communicated as one or more machine-readable files in accordance with a standardized data format (e.g., XML, JSON) to be parsed and processed by the schedule executor 1355.

The data 1370 may be received by the schedule executor 1355 from any number of sources. In some embodiments, the schedule executor 1355 may receive the data 1370 indicating performance of an activity by the patient 1350 from the client 1315. For example, the data 1370 may identify that the patient 1350 recorded eating of a healthy meal on an application on the client 1315. In some embodiments, the schedule executor 1355 may receive data 1370 including transaction data from the pharmacy service 1320 associated with the data structure 1360. For instance, the data 1370 may include transaction data identifying a transaction to receive the prescription by the patient 1350 at a value defined by the data structure 1360. In some embodiments, the schedule executor 1355 may receive data 1370 including transaction data from the care provider service 1325. For example, the data 1370 may include data identifying a refill of a prescription by a clinician associated with the care provider service 1325. In some embodiments, the schedule executor 1355 may receive the data 1370 from the program provider service 1340. For instance, the data 1370 may indicate a distribution campaign by the program provider service 1340 to credit funds to accounts corresponding to record objects 1375 associated with the patient 1350.

The schedule executor 1355 identifies or determines whether the data 1370 satisfies the target condition defined by the distribution schedule 1365 for the corresponding time point. The time point may correspond to the time point in the distribution schedule 1365 subsequent to the current time at which the data 1370 is received. The schedule executor 1355 may compare or check the data 1370 against the target condition for the time point as defined by the distribution schedule 1365. For example, if the data 1370 is received in the second month, the schedule executor 1355 may identify the target condition specified to be completed by the end of the second month in the distribution schedule 1365. Based on the comparison, the schedule executor 1355 may determine whether the data 1370 satisfies the target condition. If the data 1370 satisfies the target condition, the schedule executor 1355 may determine that the data 1370 satisfies the target condition for the corresponding time point. Otherwise, if the data 1370 does not the target condition, the schedule executor 1355 may determine that the data 1370 does not the target condition for the corresponding time point.

When the data 1370 is determined to satisfy the target condition for the corresponding time point, the schedule executor 1355 provides, sends, or otherwise transmits a request 1380 to transfer the portion of the value for the time point as defined by the distribution schedule 1365. The request 1380 may be transmitted to a transaction processor associated with the one or more record objects 1375. In some implementations, each record object 1375 may be a financial account maintained by a financial institution (e.g., a bank or brokerage) on behalf of the patient 1350, and may include, for example: a checking account, a savings account, a flexible spending account (FSA), a health savings account (HSA), a medical savings account (MSA), an employer's health reimbursement arrangement (HRA) account, or a prescription discount card, among others. The schedule executor 1355 may identify the portion of the value for the corresponding time point, as defined by the distribution schedule 1365. Upon receipt, the transaction processor may process the request 1380, and may transfer the specified amounts to the one or more record objects 1375 associated with the patient 1350. In contrast, when the data 1370 is determined to not satisfy the target condition for the corresponding time point, the schedule executor 1355 may refrain from transmitting the request 1380 to transfer the portion of the value for the time point as defined by the distribution schedule 1365.

With the transfer of the portion of the value, the schedule executor 1355 may provide, send, or otherwise transmit a notification message to the client 1315 for presentation on a user interface. The notification message may identify or indicate the corresponding portion of the value to the one or more record objects 1375 associated with the patient 1350 in accordance with the distribution schedule 1365. For instance, through the user interface, the client 1315 may display dollar amounts transferred into the record objects 1375 for fulfilling the objective as specified by the distribution schedule 1365. In some embodiments, the schedule executor 1355 may update the user profile associated with the patient 1350 to identify a remaining portion of the value available to be transferred across the one or more record objects 1375. The schedule executor 1355 may calculate or determine the remaining portion, as a function of the total value defined by the distribution schedule 1365 and the portions transferred to the record objects 1375.

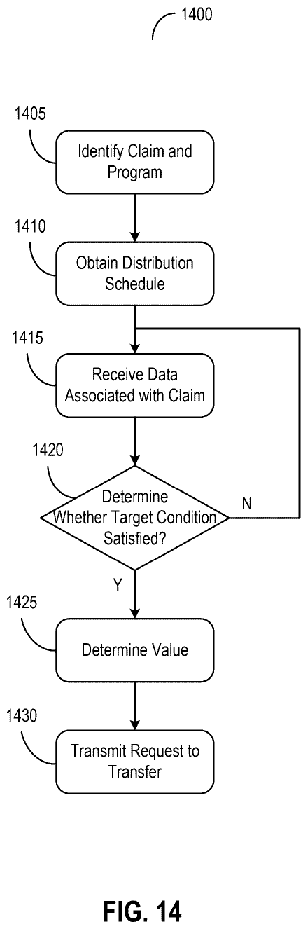

FIG. 14 depicts a flow diagram of a method 1400 of maintaining distribution schedules for data structures. The method 1400 may be implemented or performed using any of the components detailed herein. Embodiments may include additional, fewer, or different operations from those described in the method 1400. The method 1400 may be performed by a server executing machine-readable software code, though it should be appreciated that the various operations may be performed by one or more computing devices and/or processors. Under the method 1400, at step 1405, a server may identify a claim and a program. At step 1410, the server may obtain a distribution schedule. At step 1415, the server may receive data associated with the claim. At step 1420, the server may determine whether a target condition of the distribution schedule is satisfied. At step 1425, when the target condition is satisfied, the server may determine a value. At step 1430, the server may transmit a request to transfer to accounts.

The foregoing method descriptions are provided merely as illustrative examples and are not intended to require or imply that the steps of the various embodiments must be performed in the order presented. The steps in the foregoing embodiments may be performed in any order. Words such as “then,” “next,” etc., are not intended to limit the order of the steps; these words are simply used to guide the reader through the description of the methods. Many of the operations can be performed in parallel or concurrently. In addition, the order of the operations may be re-arranged. A process may correspond to a method, a function, a procedure, a subroutine, a subprogram, and the like. When a process corresponds to a function, the process termination may correspond to a return of the function to a calling function or a main function.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

The foregoing method descriptions and the process flow diagrams are provided merely as illustrative examples and are not intended to require or imply that the operations of the various embodiments must be performed in the order presented. The operations in the foregoing embodiments may be performed in any order. Words such as “then,” “next,” etc. are not intended to limit the order of the operations; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, and the like. When a process corresponds to a function, the process termination may correspond to a return of the function to a calling function or a main function.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The operations of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the generic principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A method of generating computer-readable instructions, comprising:
identifying, by a server, for a user associated with a computing device, a first data structure indicating (i) an access token to authorize intake associated with a condition in the user and (ii) a first service of a plurality of services associated with the first data structure;
receiving, by the server, a plurality of second data structures for storage on a database, each of the plurality of second data structures indicating (i) the access token and (ii) a second service of the plurality of services associated with the second data structure;
accessing, by the server, the database to retrieve a second data structure from the plurality of second data structures using the first data structure, the second data structure indicating a respective second value within a margin of a first value of the first data structure;
aggregating, by the server, on the database, data associated with the user, the first data structure, and the second data structure;
executing, by the server, a model using the data associated with the user, the first data structure, and the second data structure to generate a computer-readable instruction identifying a value for the user to accept the second data structure instead of the first data structure; and
transmitting, by the server, a notification message to the computing device for presentation on a user interface to prompt the user to accept or reject the computer-readable instruction to switch from the first data structure to the second data structure.

2. The method of claim 1, wherein accessing the database further comprises accessing the database to retrieve a subset of second data structures from the plurality of second data structures using the first data structure, each of the subset of second data structures indicating a respective second value within a margin of a first value of the first data structure,
wherein generating the computer-readable instruction further comprises generating, for each of the subset of second data structures, a respective computer-readable instruction identifying a respective value to induce the user to accept a corresponding second data structure of the subset of second data structures, and
wherein transmitting the notification message further comprises transmitting the notification message identifying the subset of second data structures and the respective computer-readable instruction for each of the subset of second data structures.

3. The method of claim 1, wherein the model further comprises a machine learning (ML) model trained using a training dataset comprising a plurality of examples, each of the plurality of examples identifying (i) a respective third data structure, (ii) a respective fourth data structure to switch with the third data structure, (ii) a respective computer-readable instruction to switch from the third data structure to the fourth data structure, and (iv) an indication of one of acceptance or rejection of the fourth data structure by a respective user.

4. The method of claim 1, wherein the model further comprises a heuristic function of the data associated with the user, the first data structure, and the second data structure to generate the computer-readable instruction to induce the user to accept the second data structure.

5. The method of claim 1, further comprising:
receiving, by the server, via the user interface from the computing device, an indication of acceptance of the computer-readable instruction; and
updating, by the server, a user account associated with the user to switch assignment from the first data structure and to the second data structure and to include an identification of the computer-readable instruction.

6. The method of claim 1, further comprising:
transmitting, by the server, responsive to the indication of the acceptance, a second notification message to a program provider service to prompt for acceptance or rejection of the computer-readable instruction;
receiving, by the server, from the program provider service, a second indication of acceptance of the program to switch to the second data structure; and
generating, by the server, in accordance with the computer-readable instruction, a distribution schedule to transfer the value to one or more accounts associated with the user.

7. The method of claim 1, further comprising:
receiving, by the server via the user interface from the computing device, an indication of a rejection of the second data structure; and
continuing, by the server, to monitor the database for a third data structure from the plurality of second data structures to switch with the first data structure, responsive to the indication of the rejection.

8. The method of claim 1, wherein identifying the first data structure further comprises receiving, from at least one of the computing device or the first service, a query identifying the first data structure for which an alternative data structure is to be identified.

9. The method of claim 1, further comprising determining, by the server, a likelihood of acceptance of switching from the first data structure to the data structure; and
wherein transmitting the notification message further comprises transmitting the notification message, responsive to an occurrence of a trigger condition corresponding to the likelihood satisfying a threshold.

10. The method of claim 1, wherein generating the computer-readable instruction further comprises generating the computer-readable instruction comprising electronic card information associated with the second data structure

11. A system for generating computer-readable instructions, comprising:
a server having one or more processors coupled with memory, configured to:
identify, for a user associated with a computing device, a first data structure indicating (i) an access token to authorize intake associated with a condition in the user and (ii) a first service of a plurality of services associated with the first data structure;
receive a plurality of second data structures for storage on a database, each of the plurality of second data structures indicating (i) the access token and (ii) a second service of the plurality of services associated with the second data structure;
access the database to retrieve a second data structure from the plurality of second data structures using the first data structure, the second data structure indicating a respective second value within a margin of a first value of the first data structure;
aggregate, on the database, data associated with the user, the first data structure, and the second data structure;
execute a model using the data associated with the user, the first data structure, and the second data structure to generate a computer-readable instruction identifying a value for the user to accept the second data structure instead of the first data structure; and
transmit a notification message to the computing device for presentation on a user interface to prompt the user to accept or reject the computer-readable instruction to switch from the first data structure to the second data structure.

12. The system of claim 11, wherein the server is further configured to:
access the database to retrieve a subset of second data structures from the plurality of second data structures using the first data structure, each of the subset of second data structures indicating a respective second value within a margin of a first value of the first data structure,
generate, for each of the subset of second data structures, a respective computer-readable instruction identifying a respective value to induce the user to accept a corresponding second data structure of the subset of second data structures, and
transmit the notification identifying the subset of second data structures and the respective computer-readable instruction for each of the subset of second data structures.

13. The system of claim 11, wherein the model further comprises a machine learning (ML) model trained using a training dataset comprising a plurality of examples, each of the plurality of examples identifying (i) a respective third data structure, (ii) a respective fourth data structure to switch with the third data structure, (ii) a respective computer-readable instruction to switch from the third data structure to the fourth data structure, and (iv) an indication of one of acceptance or rejection of the fourth data structure by a respective user.

14. The system of claim 11, wherein the model further comprises a heuristic function of the data associated with the user, the first data structure, and the second data structure to generate the computer-readable instruction to induce the user to accept the second data structure.

15. The system of claim 11, wherein the server is further configured to:
receive, via the user interface from the computing device, an indication of acceptance of the computer-readable instruction; and
update a user account associated with the user to switch assignment from the first data structure and to the second data structure and to include an identification of the computer-readable instruction.

16. The system of claim 11, wherein the server is further configured to:
transmit, responsive to the indication of the acceptance, a second notification message to a program provider service to prompt for acceptance or rejection of the computer-readable instruction;
receive, from the program provider service, a second indication of acceptance of the program to switch to the second data structure; and
generate, in accordance with the computer-readable instruction, a distribution schedule to transfer the value to one or more accounts associated with the user.

17. The system of claim 11, wherein the server is further configured to:
receive, via the user interface from the computing device, an indication of a rejection of the second data structure; and
continue to monitor the database for a third data structure from the plurality of second data structures to switch with the first data structure, responsive to the indication of the rejection.

18. The system of claim 11, wherein the server is further configured to receive, from at least one of the computing device or the first service, a query identifying the first data structure for which an alternative data structure is to be identified.

19. The system of claim 11, wherein the server is further configured to:
determine a likelihood of acceptance of switching from the first data structure to the data structure; and
transmit the notification message, responsive to an occurrence of a trigger condition corresponding to the likelihood satisfying a threshold.

20. The system of claim 11, wherein the server is further configured to generate the computer-readable instruction comprising electronic card information associated with the second data structure.
